(19) United States
(12) Patent Application Publication
(10) Pub. No.: US 2025/0186574 A1
(43) Pub. Date: Jun. 12, 2025

(54) COMPOSITIONS AND METHODS FOR THE PREVENTION AND TREATMENT OF RABIES VIRUS INFECTION

(71) Applicant: Replicate Bioscience, Inc., San Diego, CA (US)

(72) Inventors: Nathaniel Stephen WANG, San Diego, CA (US); Shigeki Joseph MIYAKE-STONER, San Diego, CA (US); Parinaz ALIAHMAD, Oakland, CA (US); Andrew GEALL, San Diego, CA (US)

(21) Appl. No.: 18/856,455

(22) PCT Filed: Apr. 12, 2023
(86) PCT No.: PCT/US23/65677

§ 371 Date: Oct. 11, 2024

(51) Int. Cl. A61K 39/205; A61K 39/00; A61P 37/04; C07K 14/005; C12N 15/86

(52) U.S. Cl. CPC A61K 39/205; A61P 37/04; C07K 14/005; C12N 15/86; A61K 2039/5256; A61K 2039/575; A61K 2039/6018; C12N 2760/20122; C12N 2760/20134; C12N 2760/20171; C12N 2770/36143

(57) ABSTRACT

The present disclosure relates generally to the field of molecular virology, and particularly relates to nucleic acid molecules encoding a modified alphavirus virus viral genome or self-replicating RNA (srRNA) construct, recombinant cells and pharmaceutical compositions containing the same, as well as the use of such nucleic acid molecules, recombinant cells and compositions for production of desired products in cell cultures or in a living body. Also provided are methods for eliciting an immune response in a subject in need thereof, as well as methods for preventing and/or treating rabies virus infection.

### CROSS-REFERENCE TO RELATED APPLICATIONS

This application claims priority to U.S. patent application Ser. No. 17/720,002, filed Apr. 13, 2022, the disclosure of which is incorporated by reference herein in its entirety, including any drawings.

### INCORPORATION BY REFERENCE OF SEQUENCE LISTING

The material in the accompanying Sequence Listing is hereby incorporated by reference into this application. The accompanying Sequence Listing file, named 2023 Apr. 6 Sequence_Listing_ST26 058462-510001WO.xml, was created on Apr. 6, 2023, and is 72,426 bytes in size.

### FIELD

The present disclosure relates to the field of molecular virology and immunology, and particularly relates to nucleic acid molecules encoding modified alphavirus viral genomes or self-replicating RNA (srRNA), recombinant cells, and pharmaceutical compositions containing the same, as well as the use of such nucleic acid molecules, recombinant cells, and compositions for production of desired products in cell cultures or in a living body. Also provided are methods for eliciting an immune response in a subject in need thereof, as well as methods for preventing and/or treating rabies virus infection.

### BACKGROUND

Rabies is a viral zoonosis, endemic in more than 100 countries and territories, and poses a threat to more than 3 billion people. The disease is invariably fatal following the onset of clinical symptoms occurring in the absence of postexposure prophylaxis (WHO epidemiological record 2010. No. 32 (85): 309-320. Rabies vaccine: WHO position paper). The WHO assumes 55000 rabies related deaths and the postexposure treatment (PET) of more than 10 million people each year.

Currently available rabies vaccines include the most widely used but highly risk-prone nerve tissue vaccines, or the safer but more costly cell culture and embryonated egg vaccines (CCEEVs). Risks associated with nerve tissue vaccines include induction of autoimmune central nervous system disease due to their inherent myelin content; the need for multiple injections; and unreliable efficacy. The WHO does not recommend the use of nerve tissue vaccines and strongly encourages the increased supply of modern and high-quality vaccines to poor populations. Avian embryo vaccines and cell culture vaccines contain inactivated purified virus, free from nerve protein. Although safer and more immunogenic than nerve tissue vaccines, cell culture production methods are time-consuming and resource-intensive and the associated cost burden largely restricts the use to the developed world despite of WHO current recommendations.

Pre-exposure prophylaxis (PrEP) with cell culture vaccine is safe and is recommended for individuals at increased risk (e.g., laboratory staff, veterinarians, animal handlers, wildlife workers and travelers to rabies-endemic areas), but it is largely restricted for reasons of cost to the developed world. Furthermore, the anti-rabies vaccine is recommended for people travelling to countries in Africa and Asia, where rabies is endemic.

A current problem is a shortage of these vaccines, which are, at certain times, only available for postexposure prophylaxis and not for prophylactic vaccination. Prophylactic vaccination is, however, important for travelers visiting developing countries where rabies virus Ig for postexposure prophylaxis may not be available.

Therefore there is a need for a safe and effective rabies vaccine which can be delivered at any time.

The disclosure provided here provides solutions to the problems existing with previous attempts to generate rabies vaccines and potentially offers improved methods for rabies infection treatment and prevention.

### SUMMARY

The present disclosure relates generally to the development of immuno-therapeutics, such as recombinant nucleic acid constructs and pharmaceutical compositions including the same for use in the prevention and management of rabies virus infection. In particular, as described in greater detail below, some embodiments of the disclosure provide nucleic acid constructs containing sequences that encode a modified alphavirus genome or self-replicating RNA (srRNA) where at least a portion of the nucleic acid sequence encoding the viral structural proteins of the modified alphavirus genome or srRNA has been replaced by a coding sequence for a polypeptide construct comprising an envelope glycoprotein G of a rabies virus (RABV-G), a variant thereof, or an antigenic determinant of either thereof. Also disclosed are recombinant cells that have been engineered to include one or more of the nucleic acid constructs disclosed herein, methods for producing a molecule of interest, and pharmaceutical compositions including one or more of the following: (a) a nucleic acid construct of the disclosure, (b) a recombinant cell of the disclosure, or (c) a pharmaceutical composition of the disclosure. Further provided in particular aspects of the disclosure are compositions and methods for eliciting an immune response in a subject in need thereof, and/or for the prevention and/or treatment of rabies virus infection. The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

In one aspect of the disclosure, provided herein are nucleic acid constructs including a nucleic acid sequence encoding a modified alphavirus genome or self-replicating RNA (srRNA), wherein at least a portion of the nucleic acid sequence encoding the viral structural proteins of the modified alphavirus genome or srRNA has been replaced by a coding sequence for a polypeptide construct comprising an envelope glycoprotein G of a rabies virus (RABV-G), a variant thereof, or an antigenic determinant of either thereof.

In some embodiments, the antigenic determinant resides within the N-terminal half of the RABV-G.

In some embodiments, the antigenic determinant resides within the C-terminal half of the RABV-G.

In some embodiments, the antigenic determinant comprises antigenic site I, antigenic site II, antigenic site III, antigenic site IV, antigenic site minor A of the RABV-G, or a combination of any thereof.

In some embodiments, the envelope glycoprotein G is of a virulent rabies virus strain or an avirulent rabies virus strain.

In some embodiments, the envelope glycoprotein G is of a rabies virus strain selected from a Flury LEP strain, a Flury LEP-C strain, a Flury HEP strain, a 1088 strain, a AT6 strain, a CQ92 strain, a CVS-11 strain, a CVS-26 strain, a CVS-26 (G-N204S) strain, a CYN1009D strain, a CYN1026D strain, a CYN1029D strain, a CYN1138D strain, a CYN1140D strain, a CYN1141D strain, a CYN1242H strain, a CYN1243D strain, a CYN1244D strain, a CYN1245D strain, a CYN1247D strain, a CYN1249D strain, a CYN1250D strain, a CYN1251D strain, a CYN1252D strain, a CYN1253D strain, a CYN1255D strain, a CYN1256D strain, a CYN1257D strain, a CYN1259D strain, a CYN1260D strain, a CYN1261D strain, a GX4 strain, a H-08-1320 strain, a H-1413-09 strain, a IP 1586/10 strain, a IP 2990/13 strain, a IP 2991/13 strain, a IP 2992/13 strain, a IP 3176/09 strain, a IP 4005/12 strain, a IP 412/10 strain, a IP 542/10 strain, a IP 7941/09 strain, a J strain, a JX-08-47 strain, a JX08-48 strain, a Kyoto strain, a Kyoto (G-S204N) strain, a N.HL strain, a RC.HL strain, a rHEP5.0-CVSG strain, a RRV ON-99-2 strain, a SAD-B19 strain, a SH06 strain, a SHRBV-18 strain, a SNK-CTN strain, a SRV9 strain, a Street Alabama Dufferin (HCP-SAD) strain, a VRC-RZ2 strain, a ZJ-LA strain, and a ZJ-QZ strain. In some embodiments, the envelope glycoprotein G is of a Flury LEP strain.

In some embodiments, the polypeptide construct comprises a molecular alteration that stabilizes the RABV-G, variant thereof, or antigenic determinant of either thereof.

In some embodiments, the modified alphavirus genome or srRNA comprises no nucleic acid sequence encoding viral structural proteins.

In some embodiments, the nucleic acid sequence encoding the polypeptide construct is operably linked to a promoter sequence. In some embodiments, the promoter sequence is a 26S subgenomic (sg) promoter.

In some embodiments, the modified alphavirus genome or srRNA is of an alphavirus belonging to the VEEV/EEEV/WEEV group, or the SFV group, or the SINV group. In some embodiments, the alphavirus is Venezuelan equine encephalitis virus (VEEV), Eastern equine encephalitis virus (EEEV), Chikungunya virus (CHIKV), Western equine encephalitis virus (WEEV), or Sindbis virus (SINV).

In some embodiments, the nucleic acid sequence has at least 80%, at least 85%, at least 90%, at least 95%, at least 96%, at least 97%, at least 98%, at least 99%, or 100% sequence identity to a nucleic acid sequence selected from the group consisting of SEQ ID NOS: 6-11.

In one aspect, provided herein are recombinant cells including a nucleic acid construct as disclosed herein. In some embodiments, the recombinant cell is a mammalian cell or an insect cell.

In yet another aspect, provided herein are pharmaceutical compositions including a pharmaceutically acceptable excipient and a nucleic acid construct of the disclosure.

In some embodiments, the composition is formulated is formulated with a delivery vehicle into a delivery system, wherein the delivery system comprises a liposome, a viral replicon particle (VRP), a lipid-based nanoparticle (LNP), a polymer nanoparticle, a physiologic buffer, a microsphere, an immune stimulating complex (ISCOM), a conjugate of bioactive ligand, or a combination of any thereof. In some embodiments, the lipid is present in mass ratio of lipid to RNA from about 100:1 to about 4:1. In some embodiments, the lipid-based nanoparticles have an average diameter of about 25 nm to about 1000 nm. In some embodiments, the composition is formulated as a vaccine or an adjuvant. In some embodiments, the composition is formulated for intramuscular administration.

In another aspect, provided herein are methods for inducing an immune response or treating rabies infection in a subject in need thereof. The method includes administering to the subject a composition comprising a nucleic acid construct of the disclosure. In some embodiments, the method is a method for inducing an immune response. In some embodiments, the immune response is a neutralizing antibody response. In some embodiments, the neutralizing antibody response comprises a neutralizing antibody titer of equal to or greater than 0.5 IU/mL. In some embodiments, the composition is administered to the subject individually as a single prophylaxis or therapy (monotherapy) or as a first therapy in combination with at least one additional therapies.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a bar chart that shows RNA replication from different srRNA vectors that encode rabies virus glycoprotein (RABV-G). srRNA was transfected at 50 or 500 ng into 7.7E5 BHK-21 cells by nucleofection and 15 hours later cells were collected and stained with an AF488-conjugated anti-dsRNA antibody. The frequency of dsRNA-positive cells was determined by flow cytometry (FC).

FIG. 2 is a bar chart that shows relative expression of rabies virus glycoprotein (RABV-G) from different srRNA vectors. srRNA encoding the RABV-G gene was transfected at 50 or 500 ng into 7.7E5 BHK-21 cells by nucleofection and 15 hours later cells were collected and stained with an AF647-conjugated anti-RabG antibody (1c5). The mean fluorescence intensity (MFI) of transfected cells was determined by flow cytometry (FC).

FIG. 3 shows srRNA-based rabies vaccines can generate T cell responses in vivo (ELISpot). T cell responses as measured by IFNγ ELISpot 14 days after a single dose of srRNA-RABV-G (0.15 ug) are shown. FIG. 3 shows the results as well as corresponding statistics on wildtype RABV-G. (**<0.01).

FIG. 4 shows srRNA-based rabies vaccines can generate protective antibody responses in vivo. Neutralizing antibody responses as measured by Rapid Fluorescent Foci Inhibition Test (RFFIT) 14 days after a single dose of srRNA-RABV-G (0.15 ug) are shown. Neutralizing antibody titers of 0.5 IU/ml, considered to be protective, are depicted by the blue line. Statistics are shown (**<0.01; *<0.05).

### DETAILED DESCRIPTION OF THE DISCLOSURE

The present disclosure relates generally to nucleic acid constructs expressing variants or antigenic determinants of RABV-G for the purposes of both prophylactic and therapeutic treatment of rabies virus infection. These constructs address the problem with current rabies vaccines, due to the requirement of a 3-dose regimen within a short time frame to elicit immune protection and the need for regular boosters due to poor durability of immune responses. Provided herein are, inter alia, gene expression systems with superior expression potential which are suitable for expressing a coding sequence for a polypeptide construct comprising an envelope glycoprotein G of a rabies virus (RABV-G), a variant thereof, or an antigenic determinant of either thereof in recombinant cells. For example, some embodiments of the disclosure relate to nucleic acid constructs such as, e.g. expression constructs and vectors, containing a modified genome or srRNA of an alphavirus in which at least a portion of the nucleic acid sequence encoding the viral structural proteins of the modified alphavirus genome or srRNA has been replaced by a coding sequence for a polypeptide construct comprising an envelope glycoprotein G of a rabies virus (RABV-G), a variant thereof, or an antigenic determinant of either thereof. Further provided are recombinant cells that are genetically engineered to include one or more of the nucleic acid molecules disclosed herein. Biomaterials and recombinant products derived from such recombinant cells are also within the scope of the application. Also provided are compositions and methods useful for eliciting an immune response or treating rabies infection in a subject in need thereof.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “construct” refers to a recombinant molecule, e.g., recombinant nucleic acid or polypeptide, including one or more nucleic acid sequences or amino acid sequences from heterologous sources. For example, polypeptide constructs can be chimeric polypeptide molecules in which two or more amino acid sequences of different origin are operably linked to one another in a single polypeptide construct. Similarly, nucleic acid constructs can be chimeric nucleic acid molecules in which two or more nucleic acid sequences of different origin are assembled into a single nucleic acid molecule. Representative nucleic acid constructs can include any recombinant nucleic acid molecules, linear or circular, single stranded or double stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Two or more nucleic acid constructs can be contained within a single nucleic acid molecule, such as a single vector, or can be containing within two or more separate nucleic acid molecules, such as two or more separate vectors.

The term “effective amount”, “therapeutically effective amount”, or “pharmaceutically effective amount” of a composition of the disclosure, e.g., nucleic acid constructs, srRNAs, recombinant cells, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “naked” as used herein when referencing nucleic acids that are substantially free of other macromolecules, such as lipids, polymers, and proteins. A “naked” nucleic acid, such as a self-replicating RNA, is not formulated with other macromolecules to improve cellular uptake. Accordingly, a naked nucleic acid is not encapsulated in, absorbed on, or bound to a liposome, a microparticle, a nanoparticle, a cationic emulsion, and the like.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “portion” as used herein refers to a fraction. With respect to a particular structure such as a polynucleotide sequence or an amino acid sequence or protein the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI website at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., rabies infection) and/or one or more symptoms of the health condition. The subject can also be an individual who is diagnosed with a risk of the health condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Alphaviruses

Alphaviruses are small, enveloped RNA viruses with a single-stranded, positive-sense RNA genome. The alphavirus genus includes, inter alia, the Sindbis virus (SINV), the Semliki Forest virus (SFV), the Ross River virus (RRV), Venezuelan equine encephalitis virus (VEEV), Eastern equine encephalitis virus (EEEV), and Western equine encephalitis virus (WEEV), which are all closely related and are able to infect various vertebrates such as mammalians, rodents, fish, avian species, and larger mammals such as humans and horses as well as invertebrates such as insects. In particular, the Sindbis and the Semliki Forest viruses have been widely studied and the life cycle, mode of replication, etc., of these viruses are well characterized.

The alphavirus genome is approximately 12 Kb long, and it consists of two open reading frames (ORFs): a 7 Kb frame encoding the nonstructural proteins (nsPs) and a 4 Kb frame encoding the structural polyprotein. The non-structural polyprotein (nsP) is cleaved into four different proteins (nsP1, nsP2, nsP3, and nsP4) which are necessary for the transcription and translation of viral mRNA inside the cytoplasm of host cells.

The nsP1 protein is an mRNA capping enzyme that possesses both guanine-7-methyltransferase (MTase) and guanylyltransferase (GTase) activities, where they direct the methylation and capping of newly synthesized viral genomic and subgenomic RNAs. The MTase motif in the N-terminal domain of nsP1 catalyzes the transfer of the methyl group from S-adenosylmethionine (AdoMet) to the N7 position of a GTP molecule (m7Gppp). GTase then binds the m7Gppp, forming a covalent link with a catalytic histidine (m7Gp-GTase) and releasing PPi. The GTase then transfers the m7Gp molecule to the 5′-diphosphate RNA to create m7GpppNp-RNA. The resulting cap structure is essential for viral mRNA translation and prevents the mRNA from being degraded by cellular 5′ exonucleases. Following the N-terminal domain are features that allow the association of the nsP1 protein to cellular membranes. The presence of α-helical amphipathic loop and palmitoylation sites allow the nsP1 protein and nsP1-containing replication complex to anchor onto the plasma membrane, possibly through nsP1 interaction with the membrane's anionic phospholipids.

The nsP2 protein possesses numerous enzymatic activities and functional roles. The N-terminal region contains a helicase domain that has seven signature motif of Superfamily 1 (SF1) helicases. It functions as an RNA triphosphatase that performs the first of the viral RNA capping reactions. It also functions as a nucleotide triphosphatase (NTPase), fueling the RNA helicase activity. The C-terminal region of nsP2 contains a papain-like cysteine protease, which is responsible for processing the viral non-structural polyprotein. The protease recognizes conserved motifs within the polyprotein. This proteolytic function is highly regulated and is modulated by other domains of nsP2. The alphavirus nsP2 protein has also been described as a virulence factor responsible for the transcriptional and translational shutoff in infected host cells and the inhibition of interferon (IFN) mediated antiviral responses contributing to the controlling of translational machinery by viral factors.

The precise role(s) of alphavirus nsP3 protein in the replication complex is less clear. The nsP3 protein has three recognized domains: the N-terminal macrodomain with phosphatase activity and nucleic acid binding ability, the alphavirus unique domain (AUD) and the C-terminal hypervariable domain. It has been demonstrated that the deletion of this domain in SFV nsP3 resulted in low viral pathogenicity, suggesting its importance in viral RNA transcription regulation.

The nsP4 polymerase is the most highly conserved protein in alphaviruses, with the most divergent being >50% identical in amino acid sequence when compared with other alphaviral nsP4s. The nsP4 contains the core RNA-dependent RNA polymerase (RdRp) domain at the C-terminal end, determined to be solely responsible for the RNA synthetic properties of the viral replication complex. The RdRp participates in replicating the genomic RNA via a negative strand RNA and transcribing the 26S subgenomic RNA. The N-terminal domain is alphavirus-specific and can be partially disordered structurally.

The 5′ two-thirds of the alphavirus genome encodes a number of non-structural proteins (nsPs) necessary for transcription and replication of viral RNA. These proteins are translated directly from the RNA and together with cellular proteins form the RNA-dependent RNA polymerase essential for viral genome replication and transcription of sgRNA. Four nsPs (nsP1-4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises sgRNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The sgRNA is transcribed from the p26S subgenomic promoter present at the 3′ end of the RNA sequence encoding the nsp4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. The sequence at the 3′ end of the genomic RNA plays an important role in the initiation negative-strand synthesis, where a minimum number of adenylate residues has been identified to be essential for replication to occur. In particular, it has been previously reported that for alphavirus genomes to replicate, there must be at least 11 residues in the poly(A) tail following the 3′ UTR to efficiently initiate minus-strand synthesis, and therefore replication to occur. It has also been previously reported that lengthening the poly(A) tail to 25 residues results in enhanced replication, but no further enhancement of replication was observed when the poly(A) was lengthened further to 34 residues. In addition, internal non-A residues in the poly(A) are most often deleterious to replication, which suggests that enzymatic poly(A) tailing would not benefit srRNA that did not exclusively contain 3′ adenylate residues following the 3′ UTR. It has been previously reported that there is no enhancement of minus-strand synthesis on RNA templates with greater than 25 adenylate residues in the poly(A) tail. In a second step of replication, the negative strand is used as a template for the production of two RNAs, respectively: (1) a positive genomic RNA corresponding to the genome of the secondary viruses producing, by translation, other nsPs and acting as a genome for the virus; and (2) sgRNA encoding the structural proteins of the virus forming the infectious particles. The positive genomic RNA/sgRNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of sgRNA is virtually exclusive, thus resulting in the production of large amount of structural protein.

### Self-Replicating RNA

As will be appreciated by the skilled artisan, the term “self-replicating RNA” refers to RNA molecule that contains all of the genetic information required for directing its own self-amplification or self-replication within a permissive cell. To direct its own replication, the srRNA generally (1) encodes polymerase, replicase, or other proteins which may interact with viral or host cell-derived proteins, nucleic acids or ribonucleoproteins to catalyze the RNA amplification process; and (2) contain cis-acting RNA sequences required for replication and transcription of the subgenomic RNA (sgRNA). These sequences may be bound during the process of replication to its self-encoded proteins, or non-self-encoded cell-derived proteins, nucleic acids or ribonucleoproteins, or complexes between any of these components. In some embodiments of the disclosure, an alphavirus srRNA construct generally contains the following elements: 5′ viral or defective-interfering RNA sequence(s) required in cis for replication, sequences coding for biologically active alphavirus non-structural proteins (e.g., nsP1, nsP2, nsP3, and nsP4), a subgenomic promoter (sg) for the sgRNA, 3′ viral sequences required in cis for replication, and optionally a polyadenylate tract (poly(A)). In some instances, a subgenomic promoter (sg) that directs expression of a heterologous sequence can be included in the srRNA construct of the disclosure.

Further, the term srRNA generally refers to a molecule of positive polarity, or “message” sense, and the srRNA may be of length different from that of any known, naturally-occurring alphavirus. In some embodiments of the present disclosure, the srRNA does not contain at least a portion of the coding sequence for one or more of the alphavirus structural proteins; and/or sequences encoding structural genes can be substituted with heterologous sequences. In those instances, where the srRNA is to be packaged into a recombinant alphavirus particle, it can contain one or more sequences, so-called packaging signals, which serve to initiate interactions with alphavirus structural proteins that lead to particle formation.

The srRNA constructs of the disclosure generally have a length of at least about 2 kb. For example, the srRNA can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the srRNA can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 Kb to about 10 kb, about 6 Kb to about 9 Kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the srRNA can have a length of about 6 kb to about 14 kb. In some embodiments, the srRNA can have a length of about 6 kb to about 16 kb.

### Rabies Virus Envelope Glycoprotein G (RABV-G)

Rabies virus is bullet shaped with a length of about 180 nm and a diameter of 75 nm. The genome is non-segmented with negative sense single stranded RNA. The genome encodes five structural proteins 3′ N-P-M-G-L 5′; N (nucleoprotein), P (phosphoprotein), M (matrix protein), G (glycoprotein) and L (RNA dependent RNA polymerase). The leader and trailer untranslated regions at the 3′ and 5′ ends of the genome flank the structural genes and between the protein encoding regions there are non-coding intergenic sequences; N-P, P-M, M-G, G-L.

Among the five structural proteins, G is present on the envelope and is the only protein exposed out. Glycoproteins are proteins which are covalently bonded with sugar units. Rabies virus glycoprotein (RABV-G) is a trimeric type I trans-membrane protein which are single pass type with extracellular N-terminus and cytoplasmic C-terminus for cell membrane. The precursor RABV-G is 524 amino acid (aa) in length (522 aa in Mokola virus), consisting of a signal peptide of 19 aa in the N terminal. The mature protein has N-terminal ectodomain (439 aa), a transmembrane segment (22 aa) and a cytoplasmic tail/endodomain/ENDO (44 aa). As the G protein covers the outer surface of the virion envelope and is the only target antigen, which is able to induce virus-neutralizing antibodies.

### Compositions of the Disclosure

As described in greater detail below, one aspect of the present disclosure relates to nucleic acid constructs containing sequences that encode a modified alphavirus genome or srRNA where at least a portion of the nucleic acid sequence encoding the viral structural proteins of the modified alphavirus genome or srRNA has been replaced by an coding sequence for a polypeptide construct comprising an envelope glycoprotein G of a rabies virus (RABV-G), a variant thereof, or an antigenic determinant of either thereof. Also provided are recombinant cells and cell cultures that have been engineered to include a nucleic acid construct as disclosed herein.

### Nucleic Acid Constructs

As described in greater detail below, one aspect of the present disclosure relates to nucleic acid constructs including a nucleic acid sequence encoding a modified genome or srRNA of an alphavirus where at least a portion of the nucleic acid sequence encoding the viral structural proteins of the modified alphavirus genome or srRNA has been replaced by a coding sequence for a polypeptide construct comprising an envelope glycoprotein G of a rabies virus (RABV-G), a variant thereof, or an antigenic determinant of either thereof. In some embodiments, the sequence encoding the nucleic acid construct can be operably linked, e.g., placed under the control of elements required for expression (e.g., promoter sequences), which allow expression of the srRNA construct in a host cell, in a subject, or in an ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

Non-limiting exemplary embodiments of the methods of the disclosure can include one or more of the following features. In some embodiments, the alphavirus srRNA vector is devoid of at least a portion of the nucleic acid sequence encoding one or more of the viral structural proteins CP, E1, E2, E3, and 6K of the alphavirus srRNA vector. In some embodiments, the alphavirus srRNA vector is devoid of a portion of or the entire sequence encoding CP. In some embodiments, the alphavirus srRNA vector is devoid of a portion of or the entire sequence encoding E1. In some embodiments, the alphavirus srRNA vector is devoid of a portion of or the entire sequence encoding E2. In some embodiments, the alphavirus srRNA vector is devoid of a portion of or the entire sequence encoding E3. In some embodiments, the alphavirus srRNA vector is devoid of a portion of or the entire sequence encoding 6K. In some embodiments, the alphavirus srRNA vector is devoid of a portion of or the entire sequence encoding a combination of CP, E1, E2, E3, and 6K. In some embodiments of the disclosure, the coding sequence for nonstructural proteins nsP1, nsP2, nsP3, and nsP4 of the alphavirus srRNA vector is present, however at least a portion of or the entire sequence encoding one or more structural proteins (e.g., CP, E1, E2, E3, and 6K) of the alphavirus srRNA vector is absent.

In some embodiments, the alphavirus srRNA vector is devoid of a substantial portion of the nucleic acid sequence encoding one or more viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full length nucleic acid sequence.

In some embodiments, the alphavirus srRNA vector is devoid of the entire sequence encoding viral structural proteins, e.g., the alphavirus srRNA vector includes no nucleic acid sequence encoding the viral structural proteins.

In some embodiments, the alphavirus srRNA vector comprises a sequence comprising CTGGAGACGTGGAGGAGAACCCTGGACCT (SEQ ID NO: 2). In some embodiments, the alphavirus vector comprises a sequence comprising GACCGCTACGCCCCAATGACCCGACCAGC (SEQ ID NO: 3). In some embodiments, the alphavirus srRNA vector comprises a sequence comprising CTGGAGACGTGGAGGAGAACCCTGGACCT (SEQ ID NO: 2) and a sequence comprising GACCGCTACGCCCCAATGACCCGACCAGC (SEQ ID NO: 3).

The nucleic acid constructs of the disclosure further include a coding sequence for a polypeptide construct that replaces at least a portion of the nucleic acid sequence encoding the viral structural proteins of the modified alphavirus genome or srRNA. In principle, the nucleic acid constructs disclosed herein can generally include any number of coding sequences for a polypeptide construct. In some embodiments, the nucleic acid constructs disclosed herein can include at least one, at least two, at least three, at least four, at least five, or at least six coding sequences for a polypeptide constructs. A coding sequence for a polypeptide construct can be a construct of genetic material that contains coding sequences and enough regulatory information to direct proper transcription and/or translation of the coding sequences in a cell, in vivo and/or ex vivo. The coding sequence for a polypeptide construct can be inserted into a vector for targeting to a desired host cell and/or into a subject. Accordingly, in some embodiments, the term “coding sequence for a polypeptide construct” can be used interchangeably with the term “expression construct.” In some embodiments, a coding sequence for a polypeptide construct can be a nucleic acid construct that includes a gene encoding a protein or functional RNA operably linked to regulatory elements such as, for example, a promoter and/or a termination signal, and optionally, any or a combination of other nucleic acid sequences that affect the transcription or translation of the gene.

The nucleic acid constructs described herein include a coding sequence for an envelope glycoprotein G of a rabies virus (RABV-G), a variant thereof, or an antigenic determinant of either thereof, which encode polypeptides containing epitopes that are able to elicit an immune response. The variants of RABV-G can encompass coding sequences for polypeptides having an amino acid sequence that is the same or essentially the same as that of the reference protein (e.g., RABV-G) except having at least one amino acid modified, for example, deleted, inserted, or replaced, respectively. The amino acid replacement may be a conservative amino acid substitution, preferably at a non-essential amino acid residue in the protein. A “conservative amino acid substitution” is one in which the amino acid residue is replaced with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A variant of a protein may have an amino acid sequence at least about 80%, 90%, 95%, or 99%, preferably at least about 90%, more preferably at least about 95%, identical to the amino acid sequence of the protein. Preferably, a variant is a functional variant of a protein that retains the same function as the protein. The terms “variant”, when used in reference to a nucleic acid sequence, refer to a nucleic acid sequence that differs by one or more nucleotides from another, usually related nucleotide acid sequence. As such, the term “variant” can refer to a change of one or more nucleotides of a reference nucleic acid which includes the insertion of one or more new nucleotides, deletion of one or more nucleotides, and substitution of one or more existing nucleotides. A variant can also include a point mutation, multiple mutation, single nucleotide polymorphism (SNP), deletion, insertion, and translocation. Thus, variants of the coding sequences described herein include nucleic acids that encode polypeptides that can be, for example, full length, mutated, truncated, inactivated, peptide/epitopes or combinations thereof of RABV-G.

The nucleic acid constructs described herein can also include a coding sequence for an envelope glycoprotein G of a rabies virus (RABV-G), or an antigenic determinant thereof. In some embodiments, the antigenic determinant resides within the N-terminal half of rabies virus G glycoprotein. Other particular antigenic determinants reside within the C-terminal half of rabies virus. Such antigenic determinants can reside, for example, within amino acids 1-50, 50-100, 100-150, 150-200, 200-250, 250-300, 300-350, 350-400, 400-450, 450-500, 500-524 of rabies virus G glycoprotein, or any interval, portion or range thereof. In one embodiment, the antigenic determinant resides within the N-terminal half of rabies virus G glycoprotein, i.e., between about amino acid residues 19-422. In another embodiment, the antigenic determinant resides within the C-terminal half of the rabies virus G glycoprotein, i.e., between about amino acid residues 1-19. In another embodiment, the antigenic determinant of the rabies G glycoprotein comprises amino acid residues 336-442. In one embodiment, the rabies G glycoprotein comprises amino acid residue 336 as well as alterations thereof, such as substitutions or deletions.

In some embodiments, the rabies G glycoprotein antigenic determinant comprises one or more epitopes. Non-limiting examples of epitopes include linear epitope, conformational epitope, discontinuous epitope, or combinations of such epitopes. One skilled in the art will understand that the term “epitope” refers to an antigenic determinant that interacts with a specific antigen-binding site of an antigen-binding polypeptide, e.g., a variable region of an antibody molecule, known as a paratope. A single antigen can have more than one epitope. Thus, different antibodies may bind to different areas on an antigen and may have different biological effects. The term “epitope” also refers to a site on an antigen to which B cells respond. It also refers to a region of an antigen that is bound by an antibody. Epitopes can be defined as structural or functional. Functional epitopes are generally a subset of the structural epitopes and have those residues that directly contribute to the affinity of the interaction. Epitopes can be linear or conformational, that is, composed of non-linear amino acids. In certain embodiments, epitopes can include determinants that are chemically active surface groupings of molecules such as amino acids, sugar side chains, phosphoryl groups, or sulfonyl groups, and, in certain embodiments, can have specific three-dimensional structural characteristics, and/or specific charge characteristics.

In another related embodiment, the rabies G glycoprotein antigenic determinant comprises or consists of antigenic site I, antigenic site II, antigenic site III, antigenic site IV, antigenic site minor A, or combinations of such antigenic sites, for example, antigenic site III and minor site A. Antigenic sites of rabies virus have been identified using panels of monoclonal antibodies and their respective monoclonal antibody-resistant virus variants. The majority of rabies virus-neutralizing monoclonal antibodies are directed against antigenic site II (Benmansour et al., “Antigenicity of Rabies Virus Glycoprotein,” J. Virol. 65:4198-4203 (1991), which is hereby incorporated by reference), which is a discontinuous conformational epitope comprising aa 34 to 42 and aa 198 to 200 (Prosniak, et al. 2003. Development of a cocktail of recombinant-expressed human rabies virus-neutralizing monoclonal antibodies for postexposure prophylaxis of rabies. J. Infect. Dis. 188:53-56). Antigenic site III is a continuous conformational epitope at aa 330 to 338 and harbors two charged residues, K330 and R333, that affect viral pathogenicity (Coulon et al 1998. An avirulent mutant of rabies virus is unable to infect motoneurons in vivo and in vitro. J. Virol. 72:273-278; Dietzschold et al. 1983. Characterization of an antigenic determinant of the glycoprotein that correlates with pathogenicity of rabies virus. Proc. Natl. Acad. Sci. USA 80:70-74; Seif et al. 1985. Rabies virulence: effect on pathogenicity and sequence characterization of rabies virus mutations affecting antigenic site III of the glycoprotein. J. Virol. 53:926-934). The conformational antigenic site I was defined by only one monoclonal antibody, 509-6, located at aa 231 (Benmansour et al., “Antigenicity of Rabies Virus Glycoprotein,” J. Virol. 65:4198-4203 (1991); Lafon et al. 1983. Antigenic sites on the CVS rabies virus glycoprotein: analysis with monoclonal antibodies. J. Gen. Virol. 64:843-8451). Antigenic site IV is known to harbor overlapping linear epitopes (Bunschoten et al., 1989. Characterization of a new virus-neutralizing epitope that denotes a sequential determinant on the rabies virus glycoprotein. J. Gen. Virol. 70:291-298; Luo et al. 1997. A virus-neutralizing epitope on the glycoprotein of rabies virus that contains Trp251 is a linear epitope. Virus Res. 51:35-41; Ni et al. 1995. Mapping and characterization of a sequential epitope on the rabies virus glycoprotein which is recognized by a neutralizing monoclonal antibody, RG719. Microbiol. Immunol. 39:693-702). Benmansour et al. also described the presence of a minor site located at position 342 to 343, which is distinct from antigenic site III despite its close proximity.

The RABV-G of the nucleic acid constructs described herein can be from a virulent rabies virus strain or an avirulent rabies virus strain.

The anti-rabies vaccines currently in use are either vaccines made from inactivated viruses or vaccines consisting of viral strains with attenuated virulence or recombinant viruses.

Viruses can be inactivated by a variety of methods, in particular chemical methods such as treatment with formaldehyde or 3-propiolactone.

The attenuation of the virulence of viral strains is well known; for example, it may be carried out by successive passages of viral strains on a different host, vector type (e.g. rabbit or mouse) or in cell cultures. Thus, strains are poorly adapted to the host source and therefore less pathogenic to the host, while maintaining their vaccination capacity.

Avirulent rabies strains often include one or more mutations in the envelope glycoprotein G (see, e.g., Flamand et al., “Avirulent Mutants of Rabies Virus and Their Use as Live Vaccine,” Trends Microbiol. 1 (8): 317-20 (1993); Coulon et al., “An Avirulent Mutant of Rabies Virus is Unable to Infect Motorneurons In Vivo and In Vitro,” J. Virol. 72 (1): 273-278 (1998)). By way of example, antigenic mutants of RABV-G isolated from either the challenge virus standard strain (CVS), CVS derivatives, or the Street-Alabama-Dufferin (SAD) Bern strain, which have the arginine (Arg) at positon 333 in G replaced by either a glutamine (Gln), a glycine (Gly), a leucine, an isoleucine, a methionine, a cysteine, or a serine, are avirulent (Tuffereau et al., Arginine or lysine in position 333 of ERA and CVS glycoprotein is necessary for rabies virulence in adult mice. Virology. 1989; 172:206-212; Seif et al., Rabies virulence: effect on pathogenicity and sequence characterization of rabies virus mutations affecting antigenic site III of the glycoprotein. J Virol. 1985; 53:926-934). In addition, recombinant viruses that have the Arg at position 333 in G replaced by a glutamic acid are also avirulent (McGettigan et al., Second-generation rabies virus-based vaccine vectors expressing human immunodeficiency virus type 1 gag have greatly reduced pathogenicity but are highly immunogenic. J Virol. 2003; 77:237-244). Thus, in general, RABV becomes avirulent when the Arg or Lys at position 333 in G is changed to other amino acids.

In principle, there are no particular limitations to the rabies virus strains from which the envelope glycoprotein G can be derived from. Non-limiting examples of rabies virus strains suitable for the compositions and methods disclosed herein include a Flury LEP strain, a Flury LEP-C strain, a Flury HEP strain, a 1088 strain, a AT6 strain, a CQ92 strain, a CVS-11 strain, a CVS-26 strain, a CVS-26 (G-N204S) strain, a CYN1009D strain, a CYN1026D strain, a CYN1029D strain, a CYN1138D strain, a CYN1140D strain, a CYN1141D strain, a CYN1242H strain, a CYN1243D strain, a CYN1244D strain, a CYN1245D strain, a CYN1247D strain, a CYN1249D strain, a CYN1250D strain, a CYN1251D strain, a CYN1252D strain, a CYN1253D strain, a CYN1255D strain, a CYN1256D strain, a CYN1257D strain, a CYN1259D strain, a CYN1260D strain, a CYN1261D strain, a GX4 strain, a H-08-1320 strain, a H-1413-09 strain, a IP 1586/10 strain, a IP 2990/13 strain, a IP 2991/13 strain, a IP 2992/13 strain, a IP 3176/09 strain, a IP 4005/12 strain, a IP 412/10 strain, a IP 542/10 strain, a IP 7941/09 strain, a J strain, a JX-08-47 strain, a JX08-48 strain, a Kyoto strain, a Kyoto (G-S204N) strain, a N.HL strain, a RC.HL strain, a rHEP5.0-CVSG strain, a RRV ON-99-2 strain, a SAD-B19 strain, a SH06 strain, a SHRBV-18 strain, a SNK-CTN strain, a SRV9 strain, a Street Alabama Dufferin (HCP-SAD) strain, a VRC-RZ2 strain, a ZJ-LA strain, and a ZJ-QZ strain. In some embodiments, the envelope glycoprotein G is of a Flury LEP strain.

The full-length amino acid sequence of a non-limiting RABV-G protein is set forth in SEQ ID NO: 1 as follows:

MVPQVLLFVPLLGFSLCFGKFPIYTIPDKLGPWSPIDIHHLSCPNNLVV
EDEGCTNLSEFSYMELKVGYISAIKVNGFTCTGVVTEAETYTNFVGYVT
TTFKRKHFRPTPDACRAAYNWKMAGDPRYEESLHNPYPDYHWLRTVKTT
KESLVIISPSVTDLDPYDKSLHSRVFPGGNCSGITVSSTYCSTNHDYTI
WMPENLRLGTSCDIFTNSRGKRASKGGKTCGFVDERGLYKSLKGACKLK
LCGVLGLRLMDGTWVAMQTSDETKWCPPGQLVNLHDFRSDEIEHLVVEE
LVKKREECLDALESIMTTKSVSFRRLSHLRKLVPGFGKAYTIFNKTLME
ADAHYKSVRTWNEIIPSKGCLRVGGRCHPHVNGVFFNGIILGSDGHVLI
PEMQSSLLQQHMELLESSVIPLMHPLADPSTVFKDGDEVEDFVEVHLPD
VHEQVSGVELGLPNWGKYVLMIAGALIALMLIIFLMTCCRRVNRPESTQ
SSLGETGRNVSVTSQSGKVISSWESYKSGGETRL

In some embodiments, the coding sequence for RABV-G in the nucleic acid constructs described herein encodes the amino acid sequence of SEQ ID NO: 1. In some embodiments, the nucleic acid constructs of the disclosure include a nucleic acid sequence encoding a RABV-G having at least 80%, at least 85%, at least 90%, at least 95%, at least 96%, at least 97%, at least 98%, at least 99%, or 100% sequence identity to an amino acid sequence of SEQ ID NO: 1. In some embodiments, the nucleic acid constructs of the disclosure include a nucleic acid sequence encoding a RABV-G having the amino acid sequence of SEQ ID NO: 1, wherein one, two, three, four, or five of the amino acid residues in a SEQ ID NO: 1 is/are substituted by a different amino acid residue. In some embodiments, the coding sequence for RABV-G encodes smaller portions of the amino acid sequence of SEQ ID NO: 1. For example, these smaller portions can include at least 8, 10, 12, 14, 16, 18, 20, 30 or more amino acids of SEQ ID NO: 1.

In some embodiments, the nucleic acid construct as described herein comprises a nucleic acid sequence has at least 80%, at least 85%, at least 90%, at least 95%, at least 96%, at least 97%, at least 98%, at least 99%, or 100% sequence identity to a nucleic acid sequence of SEQ ID NOs: 6-11.

In some embodiments, the coding sequence of RABV-G is redesigned and/or optimized for a desired property, such as increased stability, potency, and expression (e.g., translation efficiency), which in turns can maximize the impact of producing, delivering, and administering biotherapeutic. For example, in some embodiments, the coding sequence is optimized for expression at a level higher than the expression level of a reference coding sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure may also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence is optimized for expression in a target host cell through the use of codons optimized for expression. The techniques for the construction of synthetic nucleic acid sequences encoding genes using preferred codons optimal for host cell expression may be determined by computational methods analyzing the commonality of codon usage for encoding native proteins of the host cell genome and their relative abundance by techniques well known in the art. The codon usage database (http://www.kazusa.or.jp/codon) may be used for generation of codon optimized sequences in mammalian cell environments. Furthermore, a variety of software tools are available to convert sequences from one organism to the optimal codon usage for a different host organism such as the JCat Codon Optimization Tool (www.jcat.de), Integrated DNA Technologies (IDT) Codon Optimization Tool (https://www.idtdna.com/CodonOpt) or the Optimizer online codon optimization tool (http://genomes.urv.es/OPTIMIZER). Such synthetic sequences may be constructed by techniques known in the art for the construction of synthetic nucleic acid molecules and may be obtained from a variety of commercial vendors.

In some embodiments, the coding sequence is optimized for enhanced RNA stability and/or expression. The stability of RNA generally relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. Additional information regarding principles, strategies, and methods for use in enhancing RNA stability can be found at, for example, Leppek K. et al., Combinatorial optimization of mRNA structure, stability, and translation for RNA-based therapeutics. bioRxiv. (Preprint). Mar. 30, 2021. doi: 10.1101/2021.03.29.437587.

### Recombinant Cells

The nucleic acid constructs of the present disclosure can be introduced into a host cell to produce a recombinant cell containing the nucleic acid molecule. Accordingly, prokaryotic or eukaryotic cells that contain a nucleic acid construct encoding a modified alphavirus genome as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In one aspect, some embodiments of the disclosure relate to recombinant cells, for example, recombinant animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid construct present in the recombinant host cell as a mini-circle expression vector for a stable or transient expression.

In some embodiments, the recombinant cell is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell. Suitable recombinant cells include a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), a canine kidney cell (MDCK), a buffalo rat liver cell (BRL 3A), a human lung cell (W138), a human liver cell (Hep G2), a mouse mammary tumor (MMT 060562), a TRI cell, a FS4 cell, a Chinese hamster ovary cell (CHO cell), an African green monkey kidney cell (Vero cell), a human A549 cell, a human cervix cell, a human CHME5 cell, a human PER.C6 cell, a NSO murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In some embodiments, the recombinant cell is an insect cell, e.g., cell of an insect cell line. In some embodiments, the recombinant cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos.55, Sua1B, 4a-3B, Mos.43, MSQ43, and LSB-AA695BB. In some embodiments, the mosquito cell is a cell of a C6/26 cell line.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

The recombinant polypeptides produced by the method disclosed herein are also within the scope of the disclosure.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

### Pharmaceutical Compositions

The nucleic acid constructs, recombinant cells, recombinant polypeptides of the disclosure can be incorporated into compositions, including pharmaceutical compositions. Such compositions generally include one or more of the nucleic acid constructs, recombinant cells, recombinant polypeptides described and provided herein, and a pharmaceutically acceptable excipient, e.g., carrier. In some embodiments, the compositions of the disclosure are formulated for the prevention, treatment, or management of rabies infection. For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present application are formulated for use as an adjuvant.

Accordingly, in one aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. The nucleic acid constructs of the disclosure can be used in a naked form or formulated with a delivery vehicle. Exemplary routes, either using in a free form, e.g., inserted into a nucleic acid, e.g., a vector. For example, as described in greater detail below, a nucleic acid construct as described herein can be used as a vaccine.

For use in a pharmaceutical composition of the disclosure, a nucleic acid, or a recombinant cell as described herein can be formulated into or with delivery vehicles. Exemplary delivery vehicles suitable for the compositions and methods of the disclosure include, but are not limited to liposomes (e.g., neutral or anionic liposomes), microspheres, immune stimulating complexes (ISCOMS), lipid-based nanoparticles (LNP), polymer nanoparticles, viral replicon particles (VRPs), or conjugated with bioactive ligands, which can facilitate delivery and/or enhance the immune response. These compounds are readily available to one skilled in the art; for example, see Liposomes: A Practical Approach, RCP New Ed, IRL press (1990). Adjuvants other than liposomes and the like are also used and are known in the art. Adjuvants may protect the antigen (e.g., srRNA construct) from rapid dispersal by sequestering it in a local deposit, or they may contain substances that stimulate the host to secrete factors that are chemotactic for macrophages and other components of the immune system. An appropriate selection can be made by those skilled in the art, for example, from those described below.

Accordingly, in some embodiments, a composition of the disclosure can include one or more of the following: physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof.

In some embodiments, the nucleic acid constructs of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

The lipids suitable for the compositions and methods described herein can be cationic lipids, ionizable cationic lipids, anionic lipids, or neutral lipids.

In some embodiments, the LNP of the disclosure can include one or more ionizable lipids. As used herein, the term “ionizable lipid” refers to a lipid that is cationic or becomes ionizable (protonated) as the pH is lowered below the pKa of the ionizable group of the lipid, but is more neutral at higher pH values. At pH values below the pKa, the lipid is then able to associate with negatively charged nucleic acids (e.g., oligonucleotides). As used herein, the term “ionizable lipid” includes lipids that assume a positive charge on pH decrease from physiological pH, and any of a number of lipid species that carry a net positive charge at a selective pH, such as physiological pH. Permanently cationic lipids such as DOTMA have proven too toxic for clinical use. The ionizable lipid can be present in lipid formulations according to other embodiments, preferably in a ratio of about 30 to about 70 Mol %, in some embodiments, about 30 Mol %, in other embodiments, about 40 Mol %, in other embodiments, about 45 Mol % in other embodiments, about 47.5 Mol % in other embodiments, about 50 Mol %, in still other embodiments, and about 60 Mol % in yet others (“Mol %” means the percentage of the total moles that is of a particular component). The term “about” in this paragraph signifies a plus or minus range of 5 Mol %. DODMA, or 1,2-dioleyloxy-3-dimethylaminopropane, is an ionizable lipid, as is DLin-MC3-DMA or 0-(Z,Z,Z,Z-heptatriaconta-6,9,26,29-tetraen-19-yl)-4-(N,N-dimethylamino) (“MC3”).

Exemplary ionizable lipids suitable for the compositions and methods of the disclosure includes those described in PCT publications WO2020252589A1 and WO2021000041A1, U.S. Pat. Nos. 8,450,298 and 10,844,028, and Love K. T. et al., Proc Natl Acad Sci USA, Feb. 2, 2010 107 (5) 1864-1869, all of which are hereby incorporated by reference in their entirety. Accordingly, in some embodiments, the LNP of the disclosure includes one or more lipid compounds described in Love K. T. et al., 2010 supra, such as C16-96, C14-110, and C12-200. In some embodiments, the LNP includes an ionizable cationic lipid. Suitable ionizable cationic lipids include the group consisting of ALC-0315, C12-200, LN16, MC3, MD1, SM-102, and a combination of any thereof. In some embodiments, the LNP of the disclosure includes C12-200. The structure of C12-200 lipid is known in the art and described in, e.g., U.S. Pat. Nos. 8,450,298 and 10,844,028, which are hereby incorporated by reference in their entirety. In some embodiments the C12-200 is combined with cholesterol, C14-PEG2000, and DOPE. In some embodiments, the C12-200 is combined with DSPC and DMG-PEG2000.

In some embodiments, the LNP of the disclosure includes one or more cationic lipids. Suitable cationic lipids include, but are not limited to, 98N12-5, C12-200, C14-PEG2000, DLin-KC2-DMA (KC2), DLin-MC3-DMA (MC3), XTC, MD1, and 7C1. In some embodiments, the LNP of the disclosure includes one or more neutral lipids. Non-limiting neutral lipids suitable for the compositions and methods of the disclosure include DPSC, DPPC, POPC, DOPE, and SM. In some embodiments, the LNP of the disclosure includes one or more ionizable lipid compounds described in PCT publications WO2020252589A1 and WO2021000041A1, which are hereby incorporated by reference in their entirety.

A number of other lipids or combination of lipids that are known in the art can be used to produce a LNP. Non-limiting examples of lipids suitable for use to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Non-limiting examples of cationic lipids include 98N12-5, C12-200, C14-PEG2000, DLin-KC2-DMA (KC2), DLin-MC3-DMA (MC3), XTC, MD1, 7C1, and a combination of any thereof. Non-limiting examples of neutral lipids include DPSC, DPPC, POPC, DOPE, and SM. Non-limiting examples of PEG-modified lipids include PEG-DMG, PEG-CerC14, and PEG-CerC20.

In some embodiments, the LNP of the disclosure includes at least one lipid. Suitable lipids include C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). In some embodiments the C12-200 is combined with cholesterol, C14-PEG2000, and DOPE. In some embodiments, the C12-200 is combined with DSPC and DMG-PEG2000.

In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In some embodiments, the compositions of the disclosure that formulated in a liposome. In some embodiments, the compositions of the disclosure that formulated in a lipid-based nanoparticle (LNP). In some embodiments, the compositions of the disclosure that formulated in a polymer nanoparticle.

As described above, neural lipids, also known as “structural lipids” or “helper lipids” can also be incorporated into lipid formulations and lipid particles in some embodiments. The lipid formulations and lipid particles can include one or more structural lipids at about 10 to 40 Mol % of the composition. Suitable structural lipids support the formation of particles during manufacture. Structural lipids refer to any one of a number of lipid species that exist in either in an anionic, uncharged or neutral zwitterionic form at physiological pH. Representative structural lipids include diacylphosphatidylcholines, diacylphosphatidylethanolamines, diacylphosphatidylglycerols, ceramides, sphingomyelins, dihydrosphingomyelins, cephalins, and cerebrosides.

Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE).

In another embodiment, the structural lipid can be any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

Stabilizing agents can be included in lipid formulations embodiments to ensure integrity of the mixtures. Stabilizing agents are a class of molecules which disrupt or help form the hydrophobic-hydrophilic interactions among molecules. Suitable Stabilizing agents include, but are not limited to, polysorbate 80 (also known as Tween 80, IUPAC name 2-[2-[3,4-bis(2-hydroxyethoxy) oxolan-2-yl]-2-(2-hydroxyethoxy) ethoxy]ethyl octadec-9-enoate), Myrj52 (Polyoxyethylene (40) stearate), and Brij™ S10 (Polyoxyethylene (10) stearyl ether). Polyethylene glycol conjugated lipids may also be used. The stabilizing agents may be used alone or in combinations with each other.

In some embodiments, the stabilizing agents comprises about. 1 to 3 Mol % of the overall lipid mixture. In some embodiments, the stabilizing agents comprise about 0.5 to 2.5 Mol % of the overall lipid mixture. In some embodiments, the stabilizing agent is present at greater than 2.5 Mol %. In some embodiments the stabilizing agent is present at 5 Mol %. In some embodiments the stabilizing agent is present at 10 Mol %. In some embodiments, the stabilizing agent is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, and so forth. In other embodiments, the stabilizing agent is 2.6-10 Mol % of the lipid mixture. In other embodiments, the stabilizing agents is present at greater than 10 Mol % of the lipid mixture.

Steroids can also be included in the lipid compositions for certain applications, and lipid particles made therefrom include sterols, such as cholesterol and phytosterol.

In some embodiments, the therapeutic compositions described herein, e.g., nucleic acid constructs, recombinant cells, recombinant polypeptides, and/or pharmaceutical compositions are incorporated into therapeutic compositions for use in methods of preventing or treating a subject who has, who is suspected of having a rabies infection. A subject suspected of having a rabies infection can be one bitten by an animal having or suspected of having a rabies infection such as a raccoon, skunk, fox, coyote, bat, dog, cat, etc.

In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant. In some embodiments, the immunogenic compositions are formulated as a biotherapeutic, e.g., vehicle for gene delivery of different molecules with bioactivity. Non-limiting examples of biotherapeutic include cytokines, chemokines, and other soluble immunomodulators, enzymes, peptide and protein agonists, peptide and protein antagonists, hormones, receptors, antibodies and antibody-derivatives, growth factors, transcription factors, and gene silencing/editing molecules. In some embodiments, the pharmaceutical compositions are formulated as an adjuvant.

In some embodiments, the immunogenic compositions are substantially non-immunogenic or minimally immunogenic (e.g. compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intraperitoneal administration, intramuscular administration, intranodal administration, intratumoral administration, intraarticular administration, intravenous administration, subcutaneous administration, intravaginal administration, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS), tris (tromethamine), and HEPES. In these cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sucrose, trehalose, and/or sodium chloride in the composition. In some embodiments, the composition comprises tris and sucrose. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the composition is formulated for one or more of intranasal administration, transdermal administration, intramuscular administration, intranodal administration, intravenous administration, intraperitoneal administration, oral administration, or intra-cranial administration.

### Methods of the Disclosure

Administration of any one of the therapeutic compositions described herein, e.g., nucleic acid constructs, recombinant cells, recombinant polypeptides, and/or pharmaceutical compositions, can be used in the treatment of relevant health conditions, such as rabies virus infection. In some embodiments, the nucleic acid constructs, recombinant cells, recombinant polypeptides, and/or pharmaceutical compositions as described herein can be useful for eliciting an immune response in a subject in need thereof. In some embodiments, the nucleic acid constructs, recombinant cells, recombinant polypeptides, and/or pharmaceutical compositions as described herein can be incorporated into therapeutic agents for use in methods of treating a subject who has, who is suspected of having, or who may be at high risk for developing rabies infection. In some embodiments, the subject is a patient under the care of a physician.

Accordingly, in one aspect, provided herein are methods for inducing an immune response in a subject in need thereof, the method includes administering to the subject a composition including: a) a nucleic acid construct of the disclosure; b) a recombinant cell of the disclosure; c) a recombinant polypeptide of the disclosure; and/or d) a pharmaceutical composition of the disclosure.

In some embodiments, the immune response comprises a T cell response. The T cell response may comprise production of interferon γ (IFN γ). Production of IFN γ may be measured using several methods known in the art including, and without limitation, an ELISpot assay.

In some embodiments, the immune response is a neutralizing antibody response. A neutralizing antibody response is an immune response wherein specialized cells of the immune system recognize the presentation of antigen(s), and launch a specific immune response, which prevents infection of target cells from an agent, for example a virus (e.g., rabies virus). Methods of detecting neutralizing antibody responses are known in the art and may include, for example, assays as described in the Examples herein. For example, neutralizing antibody responses can be measured by Rapid Fluorescent Foci Inhibition Test (RFFIT) 14 days after a single dose of the nucleic acid construct disclosed herein. A neutralizing antibody response can also be demonstrated by injecting animals, e.g., mice or non-human primates, with the srRNA construct described herein and testing the ability of serum from the animal to neutralize the ability of the virus to infect cells. In some embodiments, the neutralizing antibody response comprises a neutralizing antibody titer of equal to or greater than 0.5 IU/mL.

In another aspect, provided herein are methods for preventing and/or treating rabies infection in a subject in need thereof, the method includes prophylactically or therapeutically administering to the subject a composition including: a) a nucleic acid construct of the disclosure; b) a recombinant cell of the disclosure; c) a recombinant polypeptide of the disclosure; and/or d) a pharmaceutical composition of any one of the disclosure.

In some embodiments, the disclosed composition is formulated to be compatible with its intended route of administration. For example, the nucleic acid constructs, recombinant cells, recombinant polypeptides, and/or pharmaceutical compositions of the disclosure may be given orally, by inhalation, or through a parenteral route. Examples of parenteral routes of administration include, for example, intramuscular, intraocular, intravenous, intranodal, intradermal, subcutaneous, transdermal (topical), transmucosal, intravaginal, and rectal administration. In some embodiments, the composition is administered intramuscularly. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates, phosphates, tris, sucrose and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

The therapeutic compositions described herein, e.g., nucleic acid constructs, recombinant cells, recombinant polypeptides, and/or pharmaceutical compositions, can be administered one from one or more times per day to one or more times per week; including once every other day. Treatment of a subject with a therapeutically effective amount of the subject nucleic acid constructs, recombinant cells, recombinant polypeptides, and/or pharmaceutical compositions of the disclosure can include a single treatment or, can include a series of treatments. In some embodiments, the compositions are administered at weekly intervals, e.g., 1 to 2, 2 to 3, or 3 to 4 doses given at 1 to 2, 2 to 3, or 3 to 4 week intervals. This may be followed with an additional administration every 1, 2, 3, or 4 months. In some embodiments, 3 doses can be administered intramuscularly at a 3 to 8 week interval, followed by intramuscular administration every 3, 6, 9, or 12 months. This may be followed with an additional administration every 1, 2, 3, or 4 years. With regard to nucleic acid constructs and recombinant polypeptides, the therapeutically effective amount of a nucleic acid construct or recombinant polypeptide of the disclosure (e.g., an effective dosage) depends on the nucleic acid construct or recombinant polypeptide selected.

As discussed supra, a therapeutically effective amount includes an amount of a therapeutic composition that is sufficient to promote a particular effect when administered to a subject, such as one who has, is suspected of having, or is at risk for a health condition, e.g., a rabies infection. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease.

A treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the ability of the compositions described herein, e.g., nucleic acid constructs, recombinant cells, recombinant polypeptides, and/or pharmaceutical compositions, to function in the treatment of rabies infection can be tested in suitable animal model systems prior to use in humans. Such animal model systems include, but are not limited to, mice, rats, hamsters, monkeys, etc. This can be measured through the ability to protect against disease and mortality from rabies virus infection by post-exposure prophylactic vaccination.

In some embodiments, a rabies virus strain as described herein is used to challenge an animal that is later administered a composition described herein, e.g., nucleic acid constructs, recombinant cells, recombinant polypeptides, and/or pharmaceutical compositions. Exemplary rabies virus strains include, without limitation, a Flury LEP strain, a Flury LEP-C strain, a Flury HEP strain, a 1088 strain, a AT6 strain, a CQ92 strain, a CVS-11 strain, a CVS-26 strain, a CVS-26 (G-N204S) strain, a CYN1009D strain, a CYN1026D strain, a CYN1029D strain, a CYN1138D strain, a CYN1140D strain, a CYN1141D strain, a CYN1242H strain, a CYN1243D strain, a CYN1244D strain, a CYN1245D strain, a CYN1247D strain, a CYN1249D strain, a CYN1250D strain, a CYN1251D strain, a CYN1252D strain, a CYN1253D strain, a CYN1255D strain, a CYN1256D strain, a CYN1257D strain, a CYN1259D strain, a CYN1260D strain, a CYN1261D strain, a GX4 strain, a H-08-1320 strain, a H-1413-09 strain, a IP 1586/10 strain, a IP 2990/13 strain, a IP 2991/13 strain, a IP 2992/13 strain, a IP 3176/09 strain, a IP 4005/12 strain, a IP 412/10 strain, a IP 542/10 strain, a IP 7941/09 strain, a J strain, a JX-08-47 strain, a JX08-48 strain, a Kyoto strain, a Kyoto (G-S204N) strain, a N.HL strain, a RC.HL strain, a rHEP5.0-CVSG strain, a RRV ON-99-2 strain, a SAD-B19 strain, a SH06 strain, a SHRBV-18 strain, a SNK-CTN strain, a SRV9 strain, a Street Alabama Dufferin (HCP-SAD) strain, a VRC-RZ2 strain, a ZJ-LA strain, and a ZJ-QZ strain. In some embodiments, the rabies virus used is Challenge Virus Standard (CVS)-11.

By way of example, the animal is infected with the rabies virus strain, and a composition described herein, e.g., nucleic acid constructs, recombinant cells, recombinant polypeptides, and/or pharmaceutical compositions, is administered on the day 0, 1, 2, 3, 4, 5, 6, 7, 8, 9, 10, 11, 12, 13, and/or 14 after infection. In some embodiments, the composition is in an LNP formulation. In some embodiments, the addition of human rabies immune globulin (HRIG) or similar rabies neutralizing immunoglobulin (e.g., equine rabies immunoglobulin, ERIG) may be combined with the srRNA treatment. In some embodiments, the recommended dose (20 IU/kg body weight of HRIG or 40 IU/kg body weight of ERIG) is administered. The animal is monitored daily for signs of disease until approximately 35 days post virus inoculation (DPI) or loss of control non-treated groups. Rabies virus infection may be monitored by real-time reverse transcriptase polymerase chain reaction (RT-qPCR). Alternatively, direct fluorescent antibody (DFA) test may be used to determine rabies virus staining in fixed tissues as per standard methodology.

In some embodiments, the nucleic acid constructs, recombinant cells, recombinant polypeptides, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments of the disclosed methods, the subject is a mammal. In some embodiments, the mammal is a human subject. As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringeability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like.

Sterile injectable solutions can be prepared by incorporating the nucleic acid constructs, recombinant cells, and/or recombinant polypeptides in the required mount in an appropriate solvent with one or a combination of ingredients enumerated above, as required, followed by filtered sterilization.

When the nucleic acid constructs, recombinant cells, recombinant polypeptides, and/or pharmaceutical compositions are suitably protected, as described above, they may be orally administered, for example, with an inert diluent or an assimilable edible carrier. The nucleic acid constructs, recombinant cells, recombinant polypeptides, and/or pharmaceutical compositions and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, suspensions, syrups, wafers, and the like.

### Additional Therapies

In some embodiments, a composition according to the present disclosure is administered to the subject individually as a single prophylaxis or therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein as well as written instructions for making and using the same. In particular, some embodiments of the disclosure provide kits for eliciting an immune response in a subject. Some other embodiments relate to kits for methods of treating cancer in a subject in need thereof. For example, provided herein, in some embodiments, are kits that include one or more of the nucleic acid constructs, recombinant cells, recombinant polypeptides, and/or pharmaceutical compositions as provided and described herein, as well as written instructions for making and using the same.

In some embodiments, the kits of the disclosure further include one or more means useful for the administration of any one of the provided nucleic acid constructs, recombinant cells, recombinant polypeptides, and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs, recombinant cells, recombinant polypeptides, and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative controls, positive controls, reagents suitable for in vitro production of the provided nucleic acid constructs, recombinant cells, recombinant polypeptides, and/or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the nucleic acid constructs, recombinant cells, recombinant polypeptides, and/or pharmaceutical compositions as provided and described herein in one container (e.g., in a sterile glass or plastic vial) and a further therapeutic agent in another container (e.g., in a sterile glass or plastic vial).

In another embodiment, the kit includes a combination of the compositions described herein, including one or more nucleic acid constructs, recombinant cells, and/or recombinant polypeptides of the disclosure in combination with one or more further therapeutic agents formulated together, optionally, in a pharmaceutical composition, in a single, common container.

If the kit includes a pharmaceutical composition for parenteral administration to a subject, the kit can include a device (e.g., an injection device or catheter) for performing such administration. For example, the kit can include one or more hypodermic needles or other injection devices as discussed above containing one or more nucleic acid constructs, recombinant cells, and/or recombinant polypeptides of the disclosure.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

Additional embodiments are disclosed in further detail in the following examples, which are provided by way of illustration and are not in any way intended to limit the scope of this disclosure or the claims.

### Example 1

Construction of Alphavirus vectors

This Example describes the experiments performed to construct base alphavirus vectors (e.g., without a heterologous gene of interest) that were subsequently used for construction of vectors that express a gene of interest (e.g., RABV-G).

### EEEV Base Vector

The base EEEV vector (i.e. without a heterologous gene of interest) was constructed as follows: The base EEEV vector was synthesized de novo in four ˜4 kb parts (Twist Bioscience) from a reference sequence (Genbank EF151502) with several modifications. Silent mutations G301A, A3550C, G4516A, G5725A, G7399A mutations were incorporated to eliminate restriction enzyme cut sites. A unique restriction enzyme cut site (SpeI, 5′-A′CTAG,T-3′) was incorporated in place of the coding sequence of the native EEEV structural genes (where the 5′ A matches the location of the structural polyprotein ATG start codon, and the 3′ T matches the location of the structural polyprotein stop codon TAA). A 5′ adaptor sequence (5′-CTGGAGACGTGGAGGAGAACCCTGGACCT-3′; SEQ ID NO: 2) was inserted upstream of the SpeI site, and a 3′ adaptor sequence (5′-GACCGCTACGCCCCAATGACCCGACCAGC-3′; SEQ ID NO: 3) was inserted downstream of the SpeI site for subsequent Gibson Assembly® procedures (Gibson et al., Nat. Methods 6, 343-345, 2009). A bacteriophage T7 RNA polymerase promoter (5′-TAATACGACTCACTATAG-3′; SEQ ID NO: 4) was included upstream of the EEEV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 5) followed by a unique restriction enzyme cut site (NotI, 5′-GC′GGCC,GC-3′). The parts were combined in a five-piece Gibson Assembly® reaction: a linearized pYL backbone and the four synthesized fragments to result in the EEEV base vector.

### CHIKV Base Vectors

The base CHIKV S27 vector was synthesized de novo in four ˜4 kb parts (Twist Bioscience, Thermo Fisher GeneArt) from a reference sequence (Genbank AF369024) with a silent A5366G mutation, and with a unique restriction enzyme cut site (SpeI, 5′-A′CTAG,T-3′) in place of the coding sequence of the CHIKV structural genes (where the 5′ A matches the location of the structural polyprotein's ATG start codon, and the 3′ T matches the location of the structural polyprotein's stop codon TAA). A 5′ adaptor sequence (5′-CTGGAGACGTGGAGGAGAACCCTGGACCT-3′; SEQ ID NO: 2) was inserted upstream of the SpeI site, and a 3′ adaptor sequence (5′-GACCGCTACGCCCCAATGACCCGACCAGC-3′; SEQ ID NO: 3) was inserted downstream of the SpeI site for subsequent Gibson Assembly® procedures. A bacteriophage T7 RNA polymerase promoter (5′-TAATACGACTCACTATAG-3′; SEQ ID NO: 4) was included upstream of the CHIKV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 5) followed by a unique restriction enzyme cut site (NotI, 5′-GC′GGCC,GC-3′). The parts were combined in a five-piece Gibson Assembly® reaction a linearized pYL backbone and the four synthesized fragments to result in the CHIKV S27 base vector.

The CHIKV DRDE base vector was similarly constructed from a reference sequence (Genbank EF210157), except the S27 3′ UTR was used in place of the DRDE 3′ UTR.

### SINV Base Vectors

The base SINV Girdwood vector was synthesized de novo in four ˜4 kb parts (Twist Bioscience, Thermo Fisher GeneArt) from a Girdwood strain reference sequence (Genbank MF459683) with a unique restriction enzyme cut site (SpeI, 5′-A′CTAG, T-3′) in place of the coding sequence of the SINV structural genes (where the 5′ A is the next nucleotide after a P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′; SEQ ID NO: 4) was included upstream of the SINNV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 5) followed by a unique restriction enzyme cut site (NotI, 5′-GC′GGCC,GC-3′). The parts were combined in a five-piece Gibson Assembly® reaction (e.g., a linearized pYL backbone and the four synthesized fragments) to result in the SINV Girdwood base vector.

The base SINV AR86 vector was similarly constructed from a reference sequence (Genbank U38305), except the nsP2 coding sequence was derived from the Girdwood reference sequence.

### VEE Base Vector

The base VEE vector was synthesized de novo in four ˜4 kb parts (Twist Bioscience, Thermo Fisher GeneArt) from a TC-83 strain reference sequence (Genbank L01443) with a silent A2087G mutation, and a unique restriction enzyme cut site (SpeI, 5′-A′CTAG,T-3′) in place of the coding sequence of the VEE structural genes (where the 5′ A is the next nucleotide after a P2A sequence following nucleotide 93 of the structural polyprotein gene, and the 3′ T matches the location of the structural polyprotein's stop codon TGA). A 5′ adaptor sequence (5′-CTGGAGACGTGGAGGAGAACCCTGGACCT-3′; SEQ ID NO: 2) was inserted upstream of the SpeI site, and a 3′ adaptor sequence (5′-GACCGCTACGCCCCAATGACCCGACCAGC-3′; SEQ ID NO: 3) was inserted downstream of the SpeI site for subsequent Gibson Assembly® procedures. A bacteriophage T7 RNA polymerase promoter (5′-TAATACGACTCACTATAG-3′; SEQ ID NO: 4) was included upstream of the VEE genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 5) followed by a unique restriction enzyme cut site (NotI, 5′-GC′GGCC,GC-3′). The parts were combined in a five-piece Gibson Assembly® reaction (e.g., a linearized pYL backbone and the four synthesized fragments) to result in the VEE base vector.

The RABV-G transgene was synthesized (IDT) with flanks homologous to the 5′ and 3′ adaptor sequences, and inserted into the SpeI-linearized base vectors by Gibson Assembly® to result in the final vectors.

### Example 2

### In Vitro Evaluation of Modified Alphaviral Vectors

This Example describes the results of in vitro experiments performed to evaluate RNA replication and RABV-G expression levels of the synthetic alphaviral srRNA constructs described in Example 1 above, and to investigate any differential behavior thereof (e.g., RNA replication and protein expression).

In vitro transcription: RNA was prepared by in vitro transcription from a SapI-linearized plasmid template with bacteriophage T7 polymerase with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA was then purified using phenol/chloroform extraction, or column purification (Monarch® RNA Cleanup Kit, NEB). RNA concentration was determined by absorbance at 260 nm (Nanodrop, Thermo Fisher Scientific).

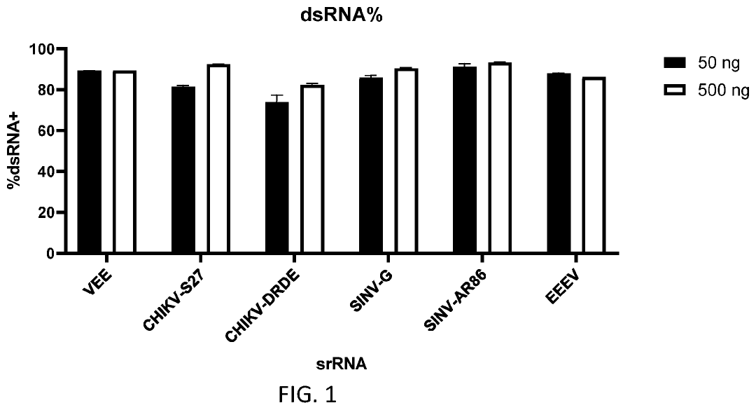

RNA replication: RNA was transformed by electroporation into BHK-21 or Vero cells (e.g., 4D-Nucleofector™, Lonza). At 15-22 hours following transformation, the cells were fixed and permeabilized (eBioscience™ Foxp3/Transcription Factor Staining Buffer Set, Invitrogen) and stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry. Results are shown in FIG. 1.

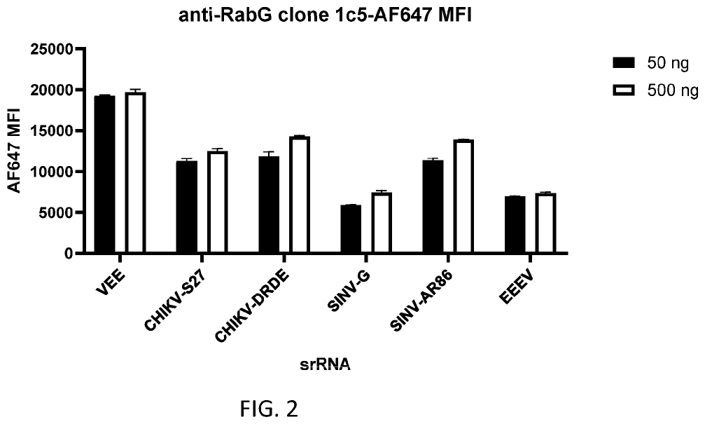

Protein expression: RNA was transformed by electroporation into BHK-21 or Vero cells (e.g., 4D-Nucleofector™, Lonza). At 15-22 hours following transformation, the cells were fixed and permeabilized (eBioscience™ Foxp3/Transcription Factor Staining Buffer Set, Invitrogen) and stained using an AF647-conjugated anti-RABV-G antibody (1c5). The mean fluorescence intensity (MFI) of AF647 was used as the readout of RABV-G expression. Results are shown in FIG. 2.

### Example 3

### In Vivo Evaluation of Modified Alphaviral Vectors

This Example describes the results of in vivo experiments performed to evaluate the srRNA constructs described herein.

In these experiments, synthetic srRNA constructs derived from various alphavirus strains were designed and subsequently evaluated.

Mice and injections. BALB/c mice were purchased from Charles River Labs, Envigo, or Jackson Laboratories. On day of dosing, 0.15 or 1.5 ug of material was injected intramuscularly either into one or split into both quadricep muscles. Vectors were LNP-formulated. Animals were monitored for body weight and other general observations throughout the course of the study. For immunogenicity studies, animals were dosed on Day 0 only or Day 0 and Day 21.

LNP formulation. srRNA was formulated in lipid nanoparticles using a microfluidics mixer and analyzed for particle size, polydispersity using dynamic light scattering and encapsulation efficiency. Lipids were suspended in ethanol. RNA was suspended in 250 mM NaOAc pH 4.0 at a concentration of 82 ug/ml, and was mixed at a flow rate of 3:1 (aqueous: organic).

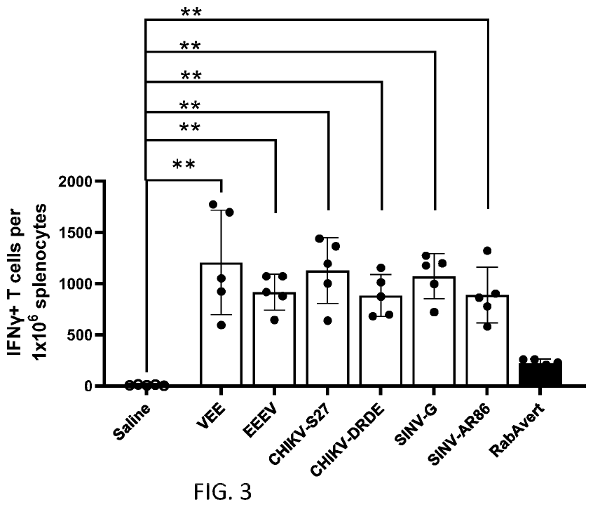

ELISpot. To measure the magnitude of RABV-G-specific T cell responses, IFNγ ELISpot analysis was performed using Mouse IFNγ ELISpot PLUS Kit (HRP) (MabTech) as per manufacturer's instructions. Results of mouse IFNγ detecting ELISpot assay as measured by spot-forming units corresponding to responder splenic T cells 14 days after intramuscular injection of srRNA encoding RABV-G is shown in FIG. 3. The total T cell responses (plotted as counted spot-forming units per million of cells) are shown the y-axis.

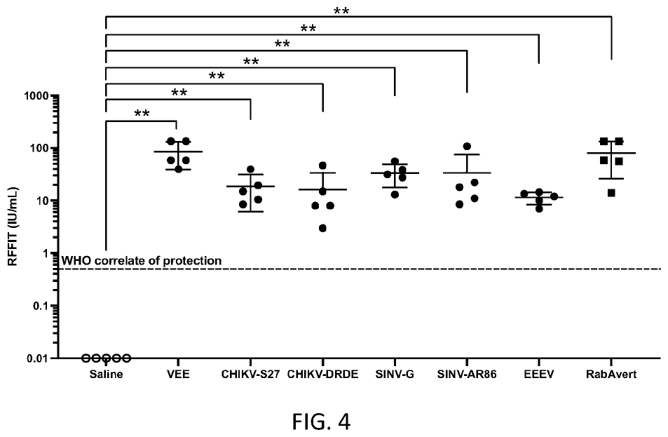

Neutralizing Antibody Response by Rapid Fluorescent Foci Inhibition Test: To measure neutralizing antibody titers generated by the vaccines, sera was obtained from mice 14 days after intramuscular administration, diluted, mixed, and incubated with a standard amount of rabies virus in multi-chambered slides. After a short incubation, cells that are susceptible to rabies virus infection and replication are added to the serum-virus mixture and incubated overnight (˜16-24 hrs) to allow for non-neutralized virus to infect the cells and replicate. The cells are then fixed and stained for rabies virus production using a fluorescent microscope. Up to a total of 20 microscopic fields are read for each serum sample and compared against a slide with a reference control serum-virus mixture. Infected cells are counted for each serum dilution and used to determine the neutralization titer. The final RFFIT result is a calculation of the counts multiplied by the dilution factor of the serum. The results of the RFFIT can be expressed as a serum titer or in International Units (IU) of antibody per milliliter of serum, the latter was done by comparing neutralizing activity of the test serum is compared to that of a reference serum with a known IU concentration (FIG. 4).

### Example 4

### In Vivo Evaluation of Modified Alphaviral Vectors as Rabies Virus Post-Exposure Prophylaxis for Treatment of Rabies Infection

This Example describes in vivo experiments performed to evaluate the srRNA constructs described herein.

In these experiments, synthetic srRNA constructs derived from various alphavirus strains are designed and subsequently evaluated for their ability to protect against disease and mortality from rabies virus infection by post-exposure prophylactic vaccination.

Challenge rabies virus. Challenge Virus Standard (CVS)-11 is purchased from American Type Culture Collection (ATCC) and is grown in baby hamster kidney (BHK)-21 cells.

LNP formulation. srRNA is formulated in lipid nanoparticles using a microfluidics mixer and analyzed for particle size, polydispersity using dynamic light scattering and encapsulation efficiency. Lipids are suspended in ethanol. RNA is suspended in 250 mM NaOAc pH 4.0 at a concentration of 82 μg/ml, and is mixed at a flow rate of 3:1 (aqueous: organic).

Mice and injections. BALB/c mice are purchased from Charles River Labs, Envigo, or Jackson Laboratories. On the day preceding srRNA dosing (Day-1), mice are intranasally inoculated with a dose of 10^{2.5} 50% cell culture infectious dose of CVS-11. On the day of srRNA dosing (Day 0, or Day 0 and Day 14), 0.1, 1, or 10 μg of material is injected intramuscularly either into one or split into both quadricep muscles. srRNAs are LNP-formulated. Animals are monitored for body weight, and clinical observations throughout the course of the study. For some groups, addition of human rabies immune globulin (HRIG) or similar rabies neutralizing immunoglobulin may be combined with the srRNA treatment.

Disease progression. Mice are observed daily for signs of disease until approximately 35 days post virus inoculation (DPI) or loss of control non-treated groups. A typical disease pattern is scored that progresses as follows: isolation from the group (score 1), slow/less vivid movement (score 2), paresis in paws (score 3), uncoordinated movement (score 4), absence of spontaneous movement (score 5), no response to stimuli (score 6) and the end-stage characterized by mice burying their heads in cage bedding and slow breathing (score 7). Disease progression is represented by plotting the daily score in function of the DPI. The incubation period is defined as the period between virus inoculation and the first appearance of disease signs. Mice are euthanized by cervical dislocation when they reach a score of 6. Results are expressed with Kaplan-Meier survival curves.

Viral load. Rabies virus infection in the brain is confirmed using real-time reverse transcriptase polymerase chain reaction (RT-qPCR). The brain is homogenized and RNA is extracted according to manufacturer's instructions (RNeasy kit, Qiagen). Alternatively, direct fluorescent antibody (DFA) test may be used to determine rabies virus staining in fixed tissues as per standard methodology.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A nucleic acid construct comprising a nucleic acid sequence encoding a modified alphavirus genome or self-replicating RNA (srRNA), wherein at least a portion of the nucleic acid sequence encoding the viral structural proteins of the modified alphavirus genome or srRNA has been replaced by a coding sequence for a polypeptide construct comprising an envelope glycoprotein G of a rabies virus (RABV-G), a variant thereof, or an antigenic determinant of either thereof.

2. The nucleic acid construct of claim 1, wherein the antigenic determinant resides within the N-terminal half of the RABV-G.

3. The nucleic acid construct of claim 1, wherein the antigenic determinant resides within the C-terminal half of the RABV-G.

4. The nucleic acid construct of claim 1, wherein the antigenic determinant comprises antigenic site I, antigenic site II, antigenic site III, antigenic site IV, antigenic site minor A of the RABV-G, or a combination of any thereof.

5. The nucleic acid construct of any preceding claim, wherein the envelope glycoprotein G is of a virulent rabies virus strain or an avirulent rabies virus strain.

6. The nucleic acid construct of any preceding claim, wherein the envelope glycoprotein G is of a rabies virus strain selected from a Flury LEP strain, a Flury LEP-C strain, a Flury HEP strain, a 1088 strain, a AT6 strain, a CQ92 strain, a CVS-11 strain, a CVS-26 strain, a CVS-26 (G-N204S) strain, a CYN1009D strain, a CYN1026D strain, a CYN1029D strain, a CYN1138D strain, a CYN1140D strain, a CYN1141D strain, a CYN1242H strain, a CYN1243D strain, a CYN1244D strain, a CYN1245D strain, a CYN1247D strain, a CYN1249D strain, a CYN1250D strain, a CYN1251D strain, a CYN1252D strain, a CYN1253D strain, a CYN1255D strain, a CYN1256D strain, a CYN1257D strain, a CYN1259D strain, a CYN1260D strain, a CYN1261D strain, a GX4 strain, a H-08-1320 strain, a H-1413-09 strain, a IP 1586/10 strain, a IP 2990/13 strain, a IP 2991/13 strain, a IP 2992/13 strain, a IP 3176/09 strain, a IP 4005/12 strain, a IP 412/10 strain, a IP 542/10 strain, a IP 7941/09 strain, a J strain, a JX-08-47 strain, a JX08-48 strain, a Kyoto strain, a Kyoto (G-S204N) strain, a N.HL strain, a RC.HL strain, a THEP5.0-CVSG strain, a RRV ON-99-2 strain, a SAD-B19 strain, a SH06 strain, a SHRBV-18 strain, a SNK-CTN strain, a SRV9 strain, a Street Alabama Dufferin (HCP-SAD) strain, a VRC-RZ2 strain, a ZJ-LA strain, and a ZJ-QZ strain.

7. The nucleic acid construct of claim 6, wherein the envelope glycoprotein G is of a Flury LEP strain.

8. The nucleic acid construct of any preceding claim, wherein the polypeptide construct comprises a molecular alteration that stabilizes the RABV-G, variant thereof, or antigenic determinant of either thereof.

9. The nucleic acid construct of any preceding claim, wherein the modified alphavirus genome or srRNA comprises no nucleic acid sequence encoding viral structural proteins.

10. The nucleic acid construct of any preceding claim, wherein the nucleic acid sequence encoding the polypeptide construct is operably linked to a promoter sequence.

11. The nucleic acid construct of claim 10, wherein the promoter sequence is a 26S subgenomic (sg) promoter.

12. The nucleic acid construct of any preceding claim, wherein the modified alphavirus genome or srRNA is of an alphavirus belonging to the VEEV/EEEV/WEEV group, or the SFV group, or the SINV group.

13. The nucleic acid of claim 12, wherein the alphavirus is Venezuelan equine encephalitis virus (VEEV), Eastern equine encephalitis virus (EEEV), Chikungunya virus (CHIKV), Western equine encephalitis virus (WEEV), or Sindbis virus (SINV).

14. The nucleic acid construct of any preceding claim, wherein the nucleic acid sequence has at least 80%, at least 85%, at least 90%, at least 95%, at least 96%, at least 97%, at least 98%, at least 99%, or 100% sequence identity to a nucleic acid sequence selected from the group consisting of SEQ ID NOS: 6-11.

15. A recombinant cell comprising a nucleic acid construct according to any one of claims 1-14.

16. The recombinant cell of claim 15, wherein the recombinant cell is a mammalian cell or an insect cell.

17. A pharmaceutical composition comprising a pharmaceutically acceptable excipient and a nucleic acid construct of any one of claims 1-14.

18. The pharmaceutical composition of claim 17, wherein the composition is formulated with a delivery vehicle into a delivery system, wherein the delivery system comprises a liposome, a viral replicon particle (VRP), a lipid-based nanoparticle (LNP), a polymer nanoparticle, a physiologic buffer, a microsphere, an immune stimulating complex (ISCOM), a conjugate of bioactive ligand, or a combination of any thereof.

19. The pharmaceutical composition of claim 18, wherein the LNP delivery system comprises a cationic lipid, an ionizable cationic lipid, an anionic lipid, or a neutral lipid.

20. The pharmaceutical composition of any one of claims 18-19, wherein the lipid is present in mass ratio of lipid to RNA from about 100:1 to about 4:1.

21. The pharmaceutical composition of any one of claims 18-20, wherein the lipid-based nanoparticles have an average diameter of about 25 nm to about 1000 nm.

22. The pharmaceutical composition of any one of claims 18-21, wherein the composition is formulated as a vaccine or an adjuvant.

23. The pharmaceutical composition of claim 17, wherein the composition is formulated for intramuscular administration.

24. A method for inducing an immune response or treating rabies infection in a subject in need thereof, the method comprises administering to the subject a composition comprising a nucleic acid construct of any one of claims 1-14.

25. The method of claim 24, wherein the method is a method for inducing an immune response.

26. The method of claim 25, wherein the immune response is a neutralizing antibody response.

27. The method of claim 26, wherein the neutralizing antibody response comprises a neutralizing antibody titer of greater than 0.5 IU/mL.

28. The method of claim 24, wherein the subject is having or suspected of having a rabies infection.

29. The method of claim 24, wherein the composition is administered to the subject individually as a single agent prophylaxis or therapy (monotherapy) or as a first therapy in combination with at least one additional therapies.


SEQUENCE LISTING (11 sequences)

SEQ ID NO: 1 - protein, 524 aa, synthetic construct
  REGION 1..524: note = Unmodified RABV-G
MVPQVLLFVPLLGFSLCFGKFPIYTIPDKLGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVTDLDPYDKSLHSRVFPGGNCSGITVSSTYCSTNHDYTIWMPENLRLGTSCDIFTNSRGKRASKGGKTCGFVDERGLYKSLKGACKLKLCGVLGLRLMDGTWVAMQTSDETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGSDGHVLIPEMQSSLLQQHMELLESSVIPLMHPLADPSTVFKDGDEVEDFVEVHLPDVHEQVSGVELGLPNWGKYVLMIAGALIALMLIIFLMTCCRRVNRPESTQSSLGETGRNVSVTSQSGKVISSWESYKSGGETRL

SEQ ID NO: 2 - DNA, 29 bp, synthetic construct
  source 1..29: mol_type = other DNA
ctggagacgtggaggagaaccctggacct

SEQ ID NO: 3 - DNA, 29 bp, synthetic construct
  source 1..29: mol_type = other DNA
gaccgctacgccccaatgacccgaccagc

SEQ ID NO: 4 - DNA, 18 bp, synthetic construct
  misc_feature 1..18: note = T7 RNA polymerase promoter
  source 1..18: mol_type = other DNA
taatacgactcactatag

SEQ ID NO: 5 - DNA, 29 bp, synthetic construct
  misc_feature 1..29: note = T7 terminator sequence
  source 1..29: mol_type = other DNA
aacccctctctaaacggaggggttttttt

SEQ ID NO: 6 - RNA, 9321 nt, synthetic construct
  misc_feature 1..9321: note = VEEV RABV-G srRNA construct
  source 1..9321: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactggtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatctggagacgtggaggagaaccctggacctatggtgcctcaggtgctgctgtttgtgcctctgctgggcttcagcctgtgcttcggcaagttccccatctacacaatccccgacaagctcggcccttggagccccatcgatatccaccacctgagctgccccaacaacctggtggtggaagatgagggctgcaccaacctgagcgagttcagctacatggaactgaaagtgggctacatcagcgccatcaaagtgaacggcttcacctgtaccggcgtggtcacagaggccgagacatacaccaacttcgtgggctacgtgaccaccaccttcaagcggaagcacttcagacccacacctgacgcctgtagagccgcctacaactggaaaatggccggcgatcccagatacgaggaaagcctgcacaacccctatcctgactaccactggctgcggaccgtgaaaaccaccaaagagagcctggtcatcatcagccccagcgtgaccgacctggatccttacgataagagcctgcactcccgggtgttccctggcggaaattgctctggcatcaccgtgtccagcacctactgcagcaccaaccacgactacaccatctggatgcccgagaacctgagactgggcaccagctgcgacatcttcaccaacagcagaggaaagcgggccagcaaaggcggcaagacctgtggctttgtggacgagagaggcctgtacaagtctctgaagggcgcctgcaagctgaagctgtgcggagttctgggactgagactgatggatggcacctgggtcgccatgcagaccagcgacgagacaaagtggtgtcctcctggccagctggtcaacctgcacgactttagatccgacgagatcgagcatctggtggtcgaggaactggtcaagaaacgggaagagtgcctggacgccctggaatccatcatgaccaccaagagcgtgtccttccggcggctgtctcacctgagaaaactggtgcctggctttggcaaggcctataccatcttcaacaagaccctgatggaagccgacgctcactacaagtctgtgcggacctggaacgagatcatccccagcaagggctgcctgagagttggcggaagatgtcaccctcacgtgaacggcgtgttcttcaacggcatcatcctgggctctgacggccacgtgctgatccctgaaatgcagtctagcctgctccagcagcatatggaactgctggaaagcagcgtgatccctctgatgcaccctctggccgatcctagcaccgtgttcaaggatggcgacgaggtcgaggacttcgtggaagtgcatctgcccgacgtgcacgagcaagtgtctggcgttgaactgggcctgcctaactggggcaaatatgtgctgatgatcgctggggccctgatcgccctgatgctgatcatcttcctgatgacctgctgtcggagagtgaacagacccgagagcacacagagcagcctgggagagacaggcagaaacgtgtccgtgacaagccagagcggcaaagtgatcagcagctgggagtcctacaagagcggcggagagacaagactgtgaccgctacgccccaatgacccgaccagctaagtaacgatacagcagcaattggcaagctgcttacatagaactcgcggcgattggcatgccgctttaaaatttttattttatttttcttttcttttccgaatcggattttgtttttaatatttc

SEQ ID NO: 7 - RNA, 9710 nt, synthetic construct
  misc_feature 1..9710: note = CHIKV-S27 RABV-G srRNA construct
  source 1..9710: mol_type = other RNA
gatggctgcgtgagacacacgtagcctaccagtttcttactgctctactctgcaaagcaagagattaagaacccatcatggatcctgtgtacgtggacatagacgctgacagcgcctttttgaaggccctgcaacgtgcgtaccccatgtttgaggtggaacctaggcaggtcacaccgaatgaccatgctaatgctagagcgttctcgcatctagctataaaactaatagagcaggaaattgatcccgactcaaccatcctggatattggtagtgcgccagcaaggaggatgatgtcggacaggaagtaccactgcgtttgcccgatgcgcagtgcagaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagatgtatcatgtagacagagagcagacgtcgcgatataccaagacgtctatgctgtacacgcacccacgtcgctataccaccaggcgattaaaggggtccgattggcgtactgggtagggtttgacacaaccccgttcatgtacaatgccatggcgggtgcctacccctcatactcgacaaattgggcagatgagcaggtactgaaggctaagaacataggattatgttcaacagacctgacggaaggtagacgaggcaaattgtctattatgagaggaaaaaagctagaaccgtgcgaccgtgtgctgttctcagtagggtcaacgctctacccggaaagccgtaagctacttaagagctggcacctaccatcggtgttccatttaaagggcaagctcagcttcacatgccgctgtgatacagtggtttcgtgcgaaggctacgtcgttaagagaataacgatgagcccaggcctttacggaaaaaccacagggtatgcggtaacccaccacgcagacggattcctgatgtgcaagaccaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaactatatgattcccgtggtcgcccaagccttcagtaagtgggcaaaggagtgccggaaagacatggaagatgaaaaactcctgggggtcagagaaagaacactgacctgctgctgtctatgggcatttaagaagcagaaaacacacacggtctacaagaggcctgatacccagtcaattcagaaggttcaggccgagtttgacagctttgtggtaccgagcctgtggtcgtccgggttgtcaatcccgttgaggactagaatcaaatggttgttaagcaaggtgccaaaaaccgacctgaccccatacagcggggacgcccaagaagcccgggacgcagaaaaagaagcagaggaagaacgagaagcagaactgactcttgaagccctaccaccccttcaggcagcacaggaagatgttcaggtcgaaatcgacgtggaacagcttgaggacagagcgggtgcaggaataatagagactccgagaggagctatcaaagttactgcccaaccaacagaccacgtcgtgggagagtacttggttctttccccgcagaccgtactacgtagccaaaagcttagcctgattcacgctttggcggagcaagtgaagacgtgcacgcacagcggacgagcagggaggtatgcggtcgaagcgtacgacggcagagtcctagtgccctcaggctacgcaatctcgcctgaagacttccagagcctaagcgaaagcgcaacgatggtgtacaacgaaagagagttcgtaaacagaaagctacaccatattgcgatgcatggaccagccctgaacaccgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatatgaagggctaaaaatccgccctgcctgcccatacaaaattgcagtcataggagtcttcggagtaccaggatctggcaagtcagctattatcaagaacctagttaccaggcaagacctggtgactagcggaaagaaagaaaactgccaagaaatcaccaccgacgtgatgagacagagaggtctagagatatctgcacgtacggttgactcgctgctcttgaatggatgtaacagaccagtcgacgtgttgtacgtagacgaggcgtttgcgtgccactctggaacgttacttgcattgatcgccttggtgagaccaagacagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgactgccattgtgtcatcgttgcattacgaaggcaaaatgcgcactacgaatgagtacaacaagccgattgtagtggacactacaggctcaacaaaacctgaccctggagatctcgtgttaacgtgcttcagaggatgggttaaacaactgcaaattgactatcgtggacacgaggtcatgacagcagccgcatcccaagggttaaccagaaaaggagtttacgcagttaggcaaaaagttaacgaaaacccgctttatgcatcaacgtcagagcacgtcaacgtactcctaacgcgtacggaaggtaaactggtatggaagacactctccggtgacccgtggataaagacgctgcagaacccaccgaaaggaaacttcaaagcaactattaaggagtgggaggtggagcatgcatcaataatggcgggcatctgcagtcaccaaatgacctttgatacattccaaaacaaagccaacgtttgttgggctaagagtttggtccctatcctcgaaacagcggggataaaactaaacgacaggcagtggtcccagataattcaagccttcaaagaagacaaagcatattcacccgaagtagccctgaatgaaatatgcacgcgcatgtatggggtggatctagacagcgggctattttctaaaccgttggtgtctgtgtattacgcggataaccactgggataataggcctggagggaagatgttcggattcaaccccgaggcagcatccattctagaaagaaagtatccatttacaaaagggaagtggaacatcaacaagcagatctgcgtgactaccaggaggatagaagacttcaaccctaccaccaacattataccggccaacaggagactaccacactcattagtggccgaacaccgcccagtaaaaggggaaagaatggaatggctggttaacaagataaacggccaccacgtgctcctggtcagtggctgtagccttgcactgcctactaagagagtcacttgggtagcgccactaggtgtccgcggagcggactatacatacaacctagagttgggtctgccagcaacgcttggtaggtatgacctagtggtcataaacatccacacaccttttcgcatacaccattatcaacagtgcgtagaccacgcaatgaaactgcaaatgctcgggggtgactcattgagactgctcaaaccgggtggctctctattgatcagagcatatggttacgcagatagaaccagtgaacgagtcatctgcgtattgggacgcaagtttagatcatctagagcgttgaaaccaccatgtgtcaccagcaacactgagatgttttttctattcagcaactttgacaatggcagaaggaatttcacaactcatgtcatgaacaatcaactgaatgcagcctttgtaggacaggccacccgagcaggatgtgcaccgtcgtaccgggtaaaacgcatggatatcgcgaagaacgatgaagagtgcgtagtcaacgccgccaaccctcgcgggttaccaggtgacggtgtttgcaaggcagtatacaaaaaatggccggagtcctttaagaacagtgcaacaccagtgggaaccgcaaaaacagtcatgtgcggtacgtatccagtaatccacgccgttggaccaaacttctctaattattcggagtctgaaggggaccgagaattggcggctgcctatcgagaagtcgcaaaggaggtaactagactgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtggagctgctggatgagcacatctccatagactgcgatgttgttcgcgtgcaccctgacagcagcttggcaggcagaaaaggatacagcaccacggaaggcgcactgtactcatatctagaagggacccgttttcaccaaacggcagtggatatggcagagatatatactatgtggccaaagcaaacagaggccaacgagcaagtttgcctatatgccctgggggaaagtattgaatcgatcaggcagaaatgcccggtggatgatgcagatgcatcatctcccccgaaaactgtcccgtgcctctgccgttacgccatgacaccagaacgcgttacccgacttcgcatgaaccatgtcacaagcataattgtgtgttcttcgtttccccttccaaagtacaaaatagaaggagtgcaaaaagtcaaatgctccaaggtaatgctatttgaccacaacgtgccatcgcgcgtaagtccaagggaatacagaccttcccaggagtctgtacaggaagcgagtacgaccacgtcactgacgcatagccaattcgatctaagcgttgacggcaagatactgcccgtcccgtcagacctggatgctgacgccccagccctagaaccagcccttgacgacggggcgatacacacgttgccatctgcaaccggaaaccttgcggccgtgtctgactgggtaatgagcaccgtacctgtcgcgccgcccagaagaaggcgagggagaaacctgactgtgacatgcgacgagagagaagggaatataacacccatggctagcgtccgattctttagggcagagctgtgtccagtcgtacaagaaacagcggagacgcgtgacacagctatgtctcttcaggcaccgccgagtaccgccacggaactgagtcacccgccgatctccttcggtgcaccaagcgagacgttccccatcacatttggggacttcaacgaaggagaaatcgaaagcttgtcttctgagctactaactttcggagacttcctacccggagaagtggatgatttgacagatagcgactggtccacgtgctcagacacggacgacgagttacgactagacagggcaggtgggtatatattctcgtcggacactggtccaggtcatttacaacagaagtcagtacgccagtcagtgctgccggtgaacaccctggaggaagtccacgaggagaagtgttacccacctaagctggatgaagcaaaggagcaactactacttaagaaactccaggagagtgcatccatggccaacagaagcaggtatcagtcgcgcaaagtagaaaacatgaaagcaacaatcatccagagactaaagagaggctgtagattatacttaatgtcagagaccccaaaagtccctacctaccggaccacatatccggcgcctgtgtactcgcctccgattaacgtccgactgtccaaccccgagtccgcagtggcagcatgcaatgagttcttggctagaaactatccaactgtttcatcataccaaatcaccgacgagtatgatgcatatctagacatggtggacgggtcggagagttgtctggaccgagcgacattcaatccgtcaaaacttaggagctacccaaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctatcaggataacaactgagaatttaacaacctatgttactaaactaaaggggccaaaagcagcagcgctatttgcaaaaacccataatctgctgccactgcaggaagtgccaatggataggttcacagtagacatgaaaagggatgtgaaggtgactcctggtacaaagcacacagaggaaagacctaaggtacaggttatacaggcggctgaacccttggcaacagcatacctatgtgggattcacagagagctggttaggaggctgaacgccgtcctcctacccaatgtacatacactatttgacatgtctgccgaggatttcgatgccatcatagccgcacactttaagccaggagacactgttttagaaacggacatagcctcctttgataagagccaagatgattcacttgcgcttactgctttaatgctgttagaggatttaggggtggatcactccctgttggacttgatagaggctgctttcggagagatttccagctgtcatctaccgacaggtacgcgcttcaagttcggcgccatgatgaaatctggtatgttcctaactctgttcgtcaacacactgctaaatatcaccatcgccagccgagtgctggaagatcgtctgacaaaatccgcgtgcgcagccttcatcggcgacgacaacataatacatggagtcgtctccgatgaattgatggcagccagatgcgccacttggatgaacatggaagtgaagatcatagatgcagttgtatcccagaaagccccttacttttgtggagggtttatactgcacgatatcgtgacaggaacagcttgcagagtggcagacccgctaaaaaggctatttaaactgggcaaaccgctagcggcaggtgacgaacaagatgaggatagaagacgagcgctggctgacgaagtggtcagatggcaacgaacagggctaattgatgagttggagaaagcggtatactctaggtatgaagtgcagggtatatcagttgtggtaatgtccatggccacctttgcaagctccagatccaacttcgagaagctcagaggacccgtcgtaactttgtacggcggtcctaaataggtacgcactacagctacctattttgcagaagccgacagtaagtacctaaacactaatcagctacactggagacgtggaggagaaccctggacctatggtgcctcaggtgctgctgtttgtgcctctgctgggcttcagcctgtgcttcggcaagttccccatctacacaatccccgacaagctcggcccttggagccccatcgatatccaccacctgagctgccccaacaacctggtggtggaagatgagggctgcaccaacctgagcgagttcagctacatggaactgaaagtgggctacatcagcgccatcaaagtgaacggcttcacctgtaccggcgtggtcacagaggccgagacatacaccaacttcgtgggctacgtgaccaccaccttcaagcggaagcacttcagacccacacctgacgcctgtagagccgcctacaactggaaaatggccggcgatcccagatacgaggaaagcctgcacaacccctatcctgactaccactggctgcggaccgtgaaaaccaccaaagagagcctggtcatcatcagccccagcgtgaccgacctggatccttacgataagagcctgcactcccgggtgttccctggcggaaattgctctggcatcaccgtgtccagcacctactgcagcaccaaccacgactacaccatctggatgcccgagaacctgagactgggcaccagctgcgacatcttcaccaacagcagaggaaagcgggccagcaaaggcggcaagacctgtggctttgtggacgagagaggcctgtacaagtctctgaagggcgcctgcaagctgaagctgtgcggagttctgggactgagactgatggatggcacctgggtcgccatgcagaccagcgacgagacaaagtggtgtcctcctggccagctggtcaacctgcacgactttagatccgacgagatcgagcatctggtggtcgaggaactggtcaagaaacgggaagagtgcctggacgccctggaatccatcatgaccaccaagagcgtgtccttccggcggctgtctcacctgagaaaactggtgcctggctttggcaaggcctataccatcttcaacaagaccctgatggaagccgacgctcactacaagtctgtgcggacctggaacgagatcatccccagcaagggctgcctgagagttggcggaagatgtcaccctcacgtgaacggcgtgttcttcaacggcatcatcctgggctctgacggccacgtgctgatccctgaaatgcagtctagcctgctccagcagcatatggaactgctggaaagcagcgtgatccctctgatgcaccctctggccgatcctagcaccgtgttcaaggatggcgacgaggtcgaggacttcgtggaagtgcatctgcccgacgtgcacgagcaagtgtctggcgttgaactgggcctgcctaactggggcaaatatgtgctgatgatcgctggggccctgatcgccctgatgctgatcatcttcctgatgacctgctgtcggagagtgaacagacccgagagcacacagagcagcctgggagagacaggcagaaacgtgtccgtgacaagccagagcggcaaagtgatcagcagctgggagtcctacaagagcggcggagagacaagactgtgaccgctacgccccaatgacccgaccagcttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 8 - RNA, 9709 nt, synthetic construct
  misc_feature 1..9709: note = CHIKV-DRDE RABV-G srRNA construct
  source 1..9709: mol_type = other RNA
gatggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgctaattatgcgagaaagctagcatctgccgcaggaaaagtcctggacagaaacatctctggaaagatcggggacttacaagcagtaatggccgtgccagacaaggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgctgtgcaagactaccgacacggttgacggcgaaagagtgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatatgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcgacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgatcgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgaccttgtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtgacggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgactgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttatgactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaagttcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtgaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtcataactttgtacggcggtcctaaataggtacgcactacagctacctattttgcagaagccgacagcaagtatctaaacactaatcagctacactggagacgtggaggagaaccctggacctatggtgcctcaggtgctgctgtttgtgcctctgctgggcttcagcctgtgcttcggcaagttccccatctacacaatccccgacaagctcggcccttggagccccatcgatatccaccacctgagctgccccaacaacctggtggtggaagatgagggctgcaccaacctgagcgagttcagctacatggaactgaaagtgggctacatcagcgccatcaaagtgaacggcttcacctgtaccggcgtggtcacagaggccgagacatacaccaacttcgtgggctacgtgaccaccaccttcaagcggaagcacttcagacccacacctgacgcctgtagagccgcctacaactggaaaatggccggcgatcccagatacgaggaaagcctgcacaacccctatcctgactaccactggctgcggaccgtgaaaaccaccaaagagagcctggtcatcatcagccccagcgtgaccgacctggatccttacgataagagcctgcactcccgggtgttccctggcggaaattgctctggcatcaccgtgtccagcacctactgcagcaccaaccacgactacaccatctggatgcccgagaacctgagactgggcaccagctgcgacatcttcaccaacagcagaggaaagcgggccagcaaaggcggcaagacctgtggctttgtggacgagagaggcctgtacaagtctctgaagggcgcctgcaagctgaagctgtgcggagttctgggactgagactgatggatggcacctgggtcgccatgcagaccagcgacgagacaaagtggtgtcctcctggccagctggtcaacctgcacgactttagatccgacgagatcgagcatctggtggtcgaggaactggtcaagaaacgggaagagtgcctggacgccctggaatccatcatgaccaccaagagcgtgtccttccggcggctgtctcacctgagaaaactggtgcctggctttggcaaggcctataccatcttcaacaagaccctgatggaagccgacgctcactacaagtctgtgcggacctggaacgagatcatccccagcaagggctgcctgagagttggcggaagatgtcaccctcacgtgaacggcgtgttcttcaacggcatcatcctgggctctgacggccacgtgctgatccctgaaatgcagtctagcctgctccagcagcatatggaactgctggaaagcagcgtgatccctctgatgcaccctctggccgatcctagcaccgtgttcaaggatggcgacgaggtcgaggacttcgtggaagtgcatctgcccgacgtgcacgagcaagtgtctggcgttgaactgggcctgcctaactggggcaaatatgtgctgatgatcgctggggccctgatcgccctgatgctgatcatcttcctgatgacctgctgtcggagagtgaacagacccgagagcacacagagcagcctgggagagacaggcagaaacgtgtccgtgacaagccagagcggcaaagtgatcagcagctgggagtcctacaagagcggcggagagacaagactgtgaccgctacgccccaatgacccgaccagcttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 9 - RNA, 9714 nt, synthetic construct
  misc_feature 1..9714: note = SINV-G RABV-G srRNA construct
  source 1..9714: mol_type = other RNA
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggtgcctcaggtgctgctgtttgtgcctctgctgggcttcagcctgtgcttcggcaagttccccatctacacaatccccgacaagctcggcccttggagccccatcgatatccaccacctgagctgccccaacaacctggtggtggaagatgagggctgcaccaacctgagcgagttcagctacatggaactgaaagtgggctacatcagcgccatcaaagtgaacggcttcacctgtaccggcgtggtcacagaggccgagacatacaccaacttcgtgggctacgtgaccaccaccttcaagcggaagcacttcagacccacacctgacgcctgtagagccgcctacaactggaaaatggccggcgatcccagatacgaggaaagcctgcacaacccctatcctgactaccactggctgcggaccgtgaaaaccaccaaagagagcctggtcatcatcagccccagcgtgaccgacctggatccttacgataagagcctgcactcccgggtgttccctggcggaaattgctctggcatcaccgtgtccagcacctactgcagcaccaaccacgactacaccatctggatgcccgagaacctgagactgggcaccagctgcgacatcttcaccaacagcagaggaaagcgggccagcaaaggcggcaagacctgtggctttgtggacgagagaggcctgtacaagtctctgaagggcgcctgcaagctgaagctgtgcggagttctgggactgagactgatggatggcacctgggtcgccatgcagaccagcgacgagacaaagtggtgtcctcctggccagctggtcaacctgcacgactttagatccgacgagatcgagcatctggtggtcgaggaactggtcaagaaacgggaagagtgcctggacgccctggaatccatcatgaccaccaagagcgtgtccttccggcggctgtctcacctgagaaaactggtgcctggctttggcaaggcctataccatcttcaacaagaccctgatggaagccgacgctcactacaagtctgtgcggacctggaacgagatcatccccagcaagggctgcctgagagttggcggaagatgtcaccctcacgtgaacggcgtgttcttcaacggcatcatcctgggctctgacggccacgtgctgatccctgaaatgcagtctagcctgctccagcagcatatggaactgctggaaagcagcgtgatccctctgatgcaccctctggccgatcctagcaccgtgttcaaggatggcgacgaggtcgaggacttcgtggaagtgcatctgcccgacgtgcacgagcaagtgtctggcgttgaactgggcctgcctaactggggcaaatatgtgctgatgatcgctggggccctgatcgccctgatgctgatcatcttcctgatgacctgctgtcggagagtgaacagacccgagagcacacagagcagcctgggagagacaggcagaaacgtgtccgtgacaagccagagcggcaaagtgatcagcagctgggagtcctacaagagcggcggagagacaagactgtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttc

SEQ ID NO: 10 - RNA, 9660 nt, synthetic construct
  misc_feature 1..9660: note = SINV-AR86 RABV-G srRNA construct
  source 1..9660: mol_type = other RNA
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctatggtgcctcaggtgctgctgtttgtgcctctgctgggcttcagcctgtgcttcggcaagttccccatctacacaatccccgacaagctcggcccttggagccccatcgatatccaccacctgagctgccccaacaacctggtggtggaagatgagggctgcaccaacctgagcgagttcagctacatggaactgaaagtgggctacatcagcgccatcaaagtgaacggcttcacctgtaccggcgtggtcacagaggccgagacatacaccaacttcgtgggctacgtgaccaccaccttcaagcggaagcacttcagacccacacctgacgcctgtagagccgcctacaactggaaaatggccggcgatcccagatacgaggaaagcctgcacaacccctatcctgactaccactggctgcggaccgtgaaaaccaccaaagagagcctggtcatcatcagccccagcgtgaccgacctggatccttacgataagagcctgcactcccgggtgttccctggcggaaattgctctggcatcaccgtgtccagcacctactgcagcaccaaccacgactacaccatctggatgcccgagaacctgagactgggcaccagctgcgacatcttcaccaacagcagaggaaagcgggccagcaaaggcggcaagacctgtggctttgtggacgagagaggcctgtacaagtctctgaagggcgcctgcaagctgaagctgtgcggagttctgggactgagactgatggatggcacctgggtcgccatgcagaccagcgacgagacaaagtggtgtcctcctggccagctggtcaacctgcacgactttagatccgacgagatcgagcatctggtggtcgaggaactggtcaagaaacgggaagagtgcctggacgccctggaatccatcatgaccaccaagagcgtgtccttccggcggctgtctcacctgagaaaactggtgcctggctttggcaaggcctataccatcttcaacaagaccctgatggaagccgacgctcactacaagtctgtgcggacctggaacgagatcatccccagcaagggctgcctgagagttggcggaagatgtcaccctcacgtgaacggcgtgttcttcaacggcatcatcctgggctctgacggccacgtgctgatccctgaaatgcagtctagcctgctccagcagcatatggaactgctggaaagcagcgtgatccctctgatgcaccctctggccgatcctagcaccgtgttcaaggatggcgacgaggtcgaggacttcgtggaagtgcatctgcccgacgtgcacgagcaagtgtctggcgttgaactgggcctgcctaactggggcaaatatgtgctgatgatcgctggggccctgatcgccctgatgctgatcatcttcctgatgacctgctgtcggagagtgaacagacccgagagcacacagagcagcctgggagagacaggcagaaacgtgtccgtgacaagccagagcggcaaagtgatcagcagctgggagtcctacaagagcggcggagagacaagactgtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttc

SEQ ID NO: 11 - RNA, 9588 nt, synthetic construct
  misc_feature 1..9588: note = EEEV RABV-G srRNA construct
  source 1..9588: mol_type = other RNA
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaaaagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagaacttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagaactggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctatggtgcctcaggtgctgctgtttgtgcctctgctgggcttcagcctgtgcttcggcaagttccccatctacacaatccccgacaagctcggcccttggagccccatcgatatccaccacctgagctgccccaacaacctggtggtggaagatgagggctgcaccaacctgagcgagttcagctacatggaactgaaagtgggctacatcagcgccatcaaagtgaacggcttcacctgtaccggcgtggtcacagaggccgagacatacaccaacttcgtgggctacgtgaccaccaccttcaagcggaagcacttcagacccacacctgacgcctgtagagccgcctacaactggaaaatggccggcgatcccagatacgaggaaagcctgcacaacccctatcctgactaccactggctgcggaccgtgaaaaccaccaaagagagcctggtcatcatcagccccagcgtgaccgacctggatccttacgataagagcctgcactcccgggtgttccctggcggaaattgctctggcatcaccgtgtccagcacctactgcagcaccaaccacgactacaccatctggatgcccgagaacctgagactgggcaccagctgcgacatcttcaccaacagcagaggaaagcgggccagcaaaggcggcaagacctgtggctttgtggacgagagaggcctgtacaagtctctgaagggcgcctgcaagctgaagctgtgcggagttctgggactgagactgatggatggcacctgggtcgccatgcagaccagcgacgagacaaagtggtgtcctcctggccagctggtcaacctgcacgactttagatccgacgagatcgagcatctggtggtcgaggaactggtcaagaaacgggaagagtgcctggacgccctggaatccatcatgaccaccaagagcgtgtccttccggcggctgtctcacctgagaaaactggtgcctggctttggcaaggcctataccatcttcaacaagaccctgatggaagccgacgctcactacaagtctgtgcggacctggaacgagatcatccccagcaagggctgcctgagagttggcggaagatgtcaccctcacgtgaacggcgtgttcttcaacggcatcatcctgggctctgacggccacgtgctgatccctgaaatgcagtctagcctgctccagcagcatatggaactgctggaaagcagcgtgatccctctgatgcaccctctggccgatcctagcaccgtgttcaaggatggcgacgaggtcgaggacttcgtggaagtgcatctgcccgacgtgcacgagcaagtgtctggcgttgaactgggcctgcctaactggggcaaatatgtgctgatgatcgctggggccctgatcgccctgatgctgatcatcttcctgatgacctgctgtcggagagtgaacagacccgagagcacacagagcagcctgggagagacaggcagaaacgtgtccgtgacaagccagagcggcaaagtgatcagcagctgggagtcctacaagagcggcggagagacaagactgtgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttc